(19) United States
(12) Patent Application Publication
(10) Pub. No.: US 2009/0270499 A1
(43) Pub. Date: Oct. 29, 2009

(54) Process for Synthesizing Atazanavir

(75) Inventor: Weiping Chen, Liverpool (GB)

Correspondence Address: FISH & RICHARDSON P.C., P.O. BOX 1022, MINNEAPOLIS, MN, 55440-1022, US

(73) Assignee: OXYRANE (UK) LIMITED, Manchester (UK)

(21) Appl. No.: 12/109,249

(22) Filed: Apr. 24, 2008

(51) Int. Cl. A61K 31/336; C07D 213/55; C07D 301/02; C07C 211/00; A61P 31/18; C07C 269/00; C07D 303/02; A61K 31/27

(52) U.S. Cl. 514/475; 546/342; 549/518; 564/305; 560/157; 549/552; 514/478

(57) ABSTRACT

This invention relates to a process for synthesizing Atazanvir, including novel intermediates and novel steps to various intermediates along the synthetic pathway.

### TECHNICAL FIELD

This invention relates to a process for synthesizing Atazanvir, including novel intermediates and novel steps to various intermediates along the synthetic pathway.

### BACKGROUND

1-[4-(Pyridin-2-yl)phenyl]-5(S)-2,5-bis{[N-(methoxycarbonyl)-L-tert-leucinyl]amino}-4(S)-hydroxy-6-phenyl-2-azahexane, Atazanavir, trade name Reyataz:

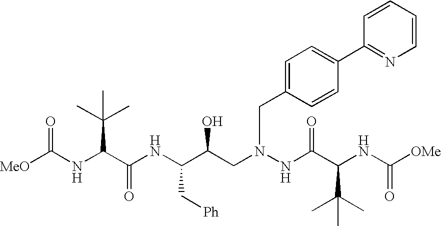

is an antiretroviral drug of the protease inhibitor class, which is used to treat infection by human immunodeficiency virus (HIV). Unlike most protease inhibitors, Atazanavir appears not to increase cholesterol, triglycerides, or blood sugar levels, which are problems to various degrees with all other protease inhibitors. Furthermore, the good oral bioavailability and favorable pharmacokinetic profile enable Atazanavir to be the first once-a-day protease inhibitor to treat AIDS (Acquired Immunodeficiency Syndrome).

A synthesis of Atazanavir was disclosed in WO 97/40029, J. Med. Chem. 1996, 39, 3203-3216, J. Med. Chem. 1998, 41, 3387-3401 and Org. Proc. Res. Dev. 2002, 6, 323-328. Several limitations of this original process presented scale-up challenges. An updated process was disclosed in WO 97/46514 and Org. Proc. Res. Dev. 2002, 12, 323-328, but scale-up challenges were still present and environmentally unfriendly solvents, such as highly flammable diethyl ether, have to be used. Although the existing processes may lead to Atazanavir, there exists a strong need to provide an alternative synthetic route to Atazanavir to ensure its manufacture in a simple and efficient manner.

### SUMMARY

Provided herein are high yielding new synthetic routes and new intermediates that provide a convenient and efficient access to Atazanavir, resulting in a product with high diastereomeric and enantiomeric purity produced in an economic manner.

Provided herein is a method of making a compound of formula (1):

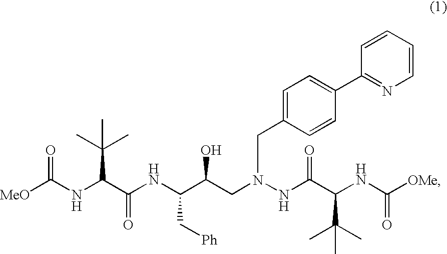

or a salt thereof, the method comprising:

a) reacting a compound of formula (12) or (13):

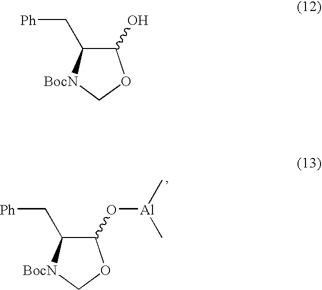

or a salt thereof, with Ar₃P═CH₂, wherein Ar is a substituted or unsubstituted C₄₋₁₂ aryl, followed by acid or base work-up, to produce a compound of formula (7):

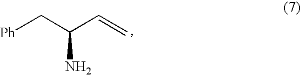

or a salt thereof;

b) coupling the compound of formula (7) with a compound of formula (8):

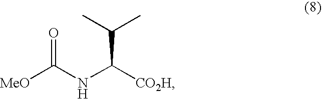

or a salt thereof, to produce a compound of formula (6):

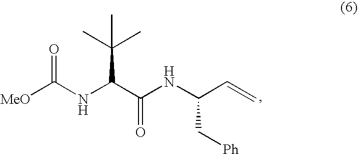

or a salt thereof;

c) reacting the compound of formula (6) with a peroxy acid, or a salt thereof, to produce a compound of formula (2):

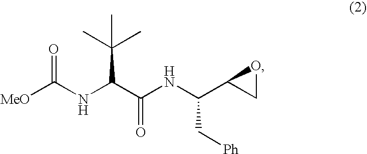

or a salt thereof;

d) reacting 4-(2-pyridyl)benzaldehyde with hydrazine hydrate in a lower alcohol to produce a compound of formula (19):

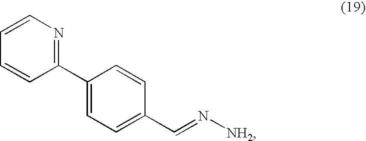

or a salt thereof;

e) coupling the compound of formula (19) with a compound of formula (8):

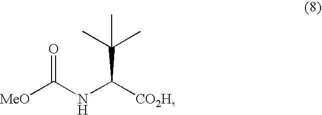

or a salt thereof, to produce a compound of formula (18):

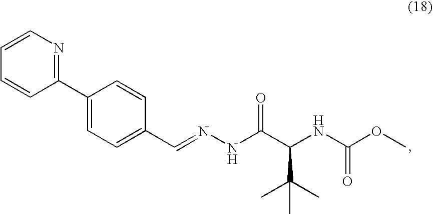

or a salt thereof;

f) hydrogenating the compound of formula (18) with a metal catalyst to produce a compound of formula (3):

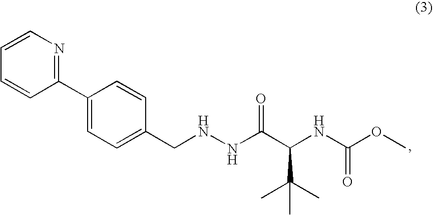

or a salt thereof; and

g) reacting the compound of formula (2), or a salt thereof, with the compound of formula (3), or a salt thereof, in the presence of a Lewis acid catalyst to make the compound of formula (1), or a salt thereof.

In some embodiments, Ar is selected from phenyl, anthracyl, and naphthyl. In some embodiments, Ar is phenyl. In some embodiments, the coupling is step (b) and (e) is performed under standard peptide formation conditions. In some embodiments, the peroxy acid, or salt thereof, is selected from the group consisting of perbenzoic acid, performic acid, peracetic acid, monoperoxyphthalic acid, pertungstic acid, m-chloroperbenzoic acid, or magnesium bis-monoperoxyphthalate hexahydrate (MMPP). n some embodiments, the metal catalyst in step (f) is selected from Pd and Ni. In some embodiments, the metal catalyst is Pd—C. In some embodiments, the Lewis acid catalyst is a heterogeneous catalyst is selected from alumina, phosphomolybdic acid-Al₂O₃, Zn(ClO₄)₂—Al₂O₃, SiO₂, heteropolyacids, zirconium sulfophenyl phosphonates, zeolites, clays, mesoporous aluminosilicates, K₅CoW₁₂O₄₀.3H₂O, (NH₄)₈[CeW₁₀O₃₆].20H₂O, Montmorillonite K-10, SBA-15, and Amberlist-15. In some embodiments, the heterogeneous catalyst is SiO₂.

In some embodiments, the compound of formula (12), or a salt thereof, is prepared by reacting a compound of formula (15):

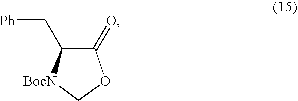

or a salt thereof, with a borohydride. In some embodiments, the compound of formula (13), or a salt thereof, is prepared by reacting a compound of formula (15):

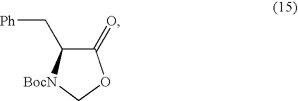

or a salt thereof, with an aluminohydride. In some embodiments, the borohydride is selected from MBH₄ or MHBR₃; wherein M is selected from Li, Na, K, Bu₄N, Zn or Ca; and R is a C₁₋₁₀ alkyl. In some embodiments, the borohydride is selected from LiBH₄, NaBH₄, or KBH₄. In some embodiments, the aluminohydride is selected from HAlR₂, MHAl(OR)₃, and MH₂Al(OR)₂; wherein M is selected from Li, Na, K, Bu₄N, Zn or Ca; and R is a C₁₋₁₀ alkyl. In some embodiments, the aluminohydride is selected from HAl(Bu-i)₂, LiHAl(O-Bu-t)₃, LiHAl(OMe)₃, LiHAl(OEt)₃, and NaH₂Al(OCH₂CH₂OCH₃)₂.

Also provided herein is a method of making a compound of formula (1):

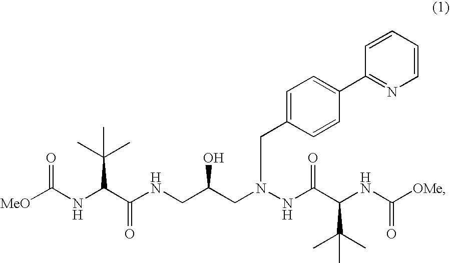

or a salt thereof, the method comprising:

a) reacting a compound of formula (13):

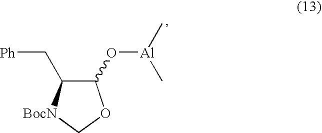

or a salt thereof, with Ph₃P═CH₂ with hydrochloric acid, to produce a compound of formula (7):

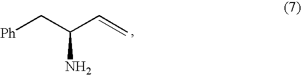

or a salt thereof;

b) coupling the compound of formula (7) with a compound of formula (8):

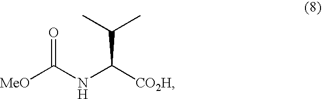

or a salt thereof, to produce a compound of formula (6):

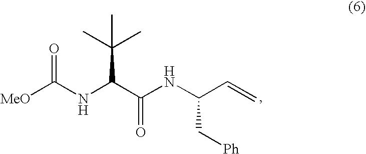

or a salt thereof;

c) reacting the compound of formula (6) with magnesium bis-monoperoxyphthalate hexahydrate (MMPP), to produce a compound of formula (2):

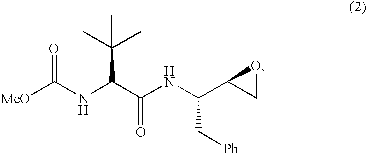

or a salt thereof;

d) reacting 4-(2-pyridyl)benzaldehyde with hydrazine hydrate in a lower alcohol to produce a compound of formula (19):

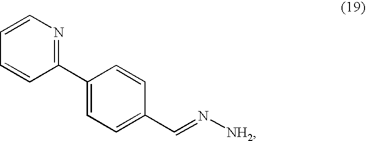

or a salt thereof;

e) coupling the compound of formula (19) with a compound of formula (8):

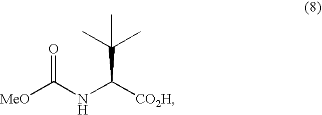

or a salt thereof, to produce a compound of formula (18):

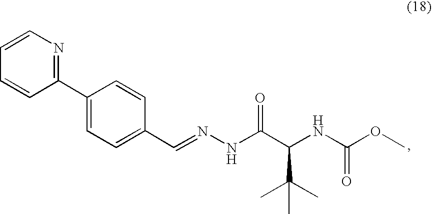

or a salt thereof;

f) hydrogenating the compound of formula (18) with a formate and Pd—C to produce a compound of formula (3):

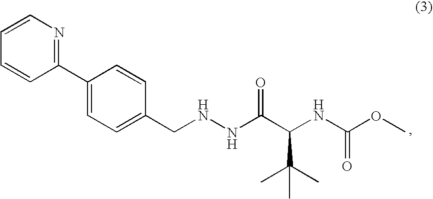

or a salt thereof; and

g) reacting the compound of formula (2), or a salt thereof, with the compound of formula (3), or a salt thereof, in the presence of SiO₂ to make the compound of formula (1), or a salt thereof.

In some embodiments, the formate is selected from sodium formate, potassium formate, and ammonium formate.

Further provided herein is a method of making a compound of formula (1):

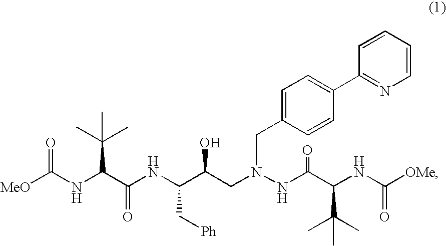

or a salt thereof, the method comprising reacting a compound of formula (2):

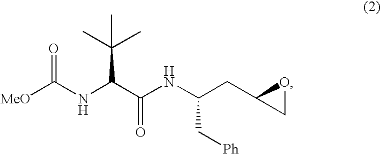

or a salt thereof, with a compound of formula (3):

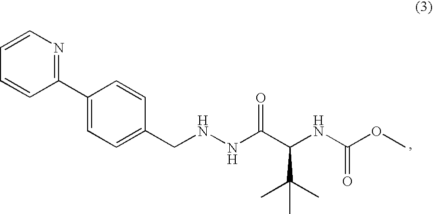

or a salt thereof, in the presence of a Lewis acid catalyst and an inert solvent thereby making the compound of formula (1), or a salt thereof.

In some embodiments, the Lewis acid catalyst is selected from the group consisting of: a metal triflate, metal halide, metal perchlorate, metal tetrafluoroborate, fluoroalkyl alcohol, and a heterogeneous catalyst. Non-limiting examples of the metal triflate include Li(OTf), Sn(OTf)₂, Cu(OTf)₂, Bi(OTf)₃, Ca(OTf)₂, Al(OTf)₃, Sm(OTf)₃, Yb(OTf)₃, and Sc(OTf)₃. Non-limiting examples of the metal halide include CeCl₃, WCl₆, ZrCl₄, RuCl₃, SbCl₃, CoCl₂, CdCl₂, TaCl₅, InCl₃, BiCl₃, VCl₃, SnCl₄, ZnCl₂, ZrCl₄, InBr₃, MgBr₂, LiBr, SmI₂, and SmCl₃. Non-limiting examples of the metal perchlorate include LiClO₄, NaClO₄, Zn(ClO₄)₂, and Cu(ClO₄)₂. In some embodiments, the fluoroalkyl alcohol is hexafluoro-2-propanol. Non-limiting examples of the heterogeneous catalyst is selected from the group consisting of: alumina, phosphomolybdic acid-Al₂O₃, Zn(ClO₄)₂—Al₂O₃, SiO₂, heteropolyacids, zirconium sulfophenyl phosphonates, zeolites, clays, mesoporous aluminosilicates, K₅CoW₁₂O₄₀.3H₂O, (NH₄)₈[CeW₁₀O₃₆].20H₂O, Montmorillonite K-10, SBA-15, and Amberlist-15. In some embodiments, the Lewis acid catalyst is SiO₂.

In some embodiments, the inert solvent is selected from the group consisting of: an alcohol, ether, amide, ester, chlorinated hydrocarbon, hydrocarbon, or mixtures thereof. Non-limiting examples of the alcohol include methanol, ethanol, n-propanol, isopropanol, n-butanol, isobutanol, tert-butanol, and ethylene glycol. Non-limiting examples of the ether include diethyl ether, diisopropyl ether, tetrahydrofuran, and dioxane. Non-limiting examples of the amide include dimethylformamide and dimethylacetamide. Non-limiting examples of the ester include ethyl acetate, methyl acetate and ethyl formate. Non-limiting examples of the chlorinated hydrocarbon include dichloromethane, chloroform, and dichloroethane. Non-limiting examples of the hydrocarbon include hexane, heptane, benzene, and toluene. In some embodiments, the inert solvent is dichloromethane.

A method of making a compound of formula (4):

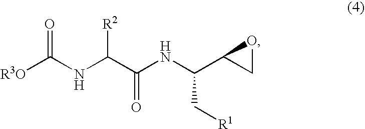

or a salt thereof, is provided wherein:

R¹ is selected from C₄₋₁₀ alkyl, C₅₋₁₂ cycloalkyl, C₅₋₁₂ heterocycloalkyl, C₅₋₁₂ aryl, C₅₋₁₂ heteroaryl, and an amino acid side chain, wherein the alkyl, cycloalkyl, heterocycloalkyl, aryl and heteroaryl can be substituted or unsubstituted; and

R² is selected from H, C₁₋₁₀ substituted or unsubstituted alkyl, and an amino acid side chain; and

R³ is selected from H and C₁₋₁₀ substituted or unsubstituted alkyl;

the method comprising: reacting a compound of formula (5):

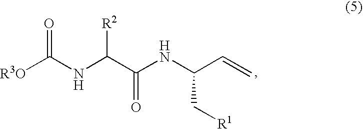

or a salt thereof, wherein R¹, R², and R³ are as defined above;
with one or more of:

a) molecular oxygen or air in the presence of a metal catalyst;

b) hydrogen peroxide or an organic peroxide in the presence of a metal catalyst; or

c) a peroxy acid, or a salt thereof;

in a solvent, thereby making a compound of formula (4), or a salt thereof.

In some embodiments, the metal catalyst is selected from: a transition metal oxide solid acid (TMO); tungstic acid or its derivatives; vanadium complexes; molybdenum complexes; titanium complexes; and cobalt complexes.

In some embodiments, the peroxy acid, or salt thereof, is selected from the group consisting of perbenzoic acid, performic acid, peracetic acid, monoperoxyphthalic acid, pertungstic acid, m-chloroperbenzoic acid, or magnesium bis-monoperoxyphthalate hexahydrate (MMPP). In some embodiments, the peroxy acid is MMPP.

In some embodiments, the solvent is selected from the group consisting of: alcohols, ethers, or chlorinated hydrocarbons. In some embodiments, the solvent is dichloromethane. In some embodiments, the solvent is methanol.

In some embodiments, R¹ is a C₅₋₁₂ aryl. In some embodiments, R¹ is an amino acid side chain selected from a side chain of histidine, phenylalanine, tryptophan, and tyrosine. In some embodiments, R¹ is phenyl. In some embodiments, R² is an amino acid side chain. In some embodiments, R² is a C₁₋₁₀ alkyl. In some embodiments, R² is tert-butyl. In some embodiments, R³ is a C₁₋₁₀ alkyl. In some embodiments, R³ is methyl. In some embodiments, the compound of formula (4) is:

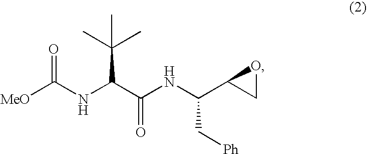

or a salt thereof.

Provided herein is a method of making a compound of formula (2):

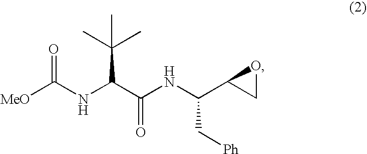

or a salt thereof, the method comprising reacting a compound of formula (6):

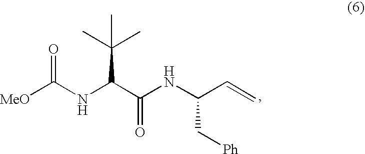

or a salt thereof, with one or more of:

a) molecular oxygen or air in the presence of a metal catalyst;

b) hydrogen peroxide or an organic peroxide in the presence of a metal catalyst; or

c) a peroxy acid, or a salt thereof;

in a solvent, thereby making a compound of formula (2), or a salt thereof.

Also provided herein is a method of making a compound of formula (16):

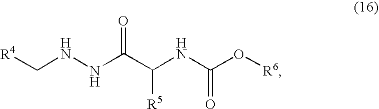

or a salt thereof, wherein:

R⁴ is selected from C₄₋₁₀ alkyl, C₅₋₁₂ cycloalkyl, C₅₋₁₂ heterocycloalkyl, C₅₋₁₂ aryl, C₅₋₁₂ heteroaryl, and an amino acid side chain, wherein the alkyl, cycloalkyl, heterocycloalkyl, aryl and heteroaryl can be substituted or unsubstituted; and

R⁵ is selected from H, C₁₋₁₀ substituted or unsubstituted alkyl, and an amino acid side chain; and

R⁶ is selected from H and C₁₋₁₀ substituted or unsubstituted alkyl;

the method comprising reacting a compound of formula (17):

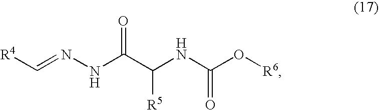

or a salt thereof, wherein R⁴, R⁵, and R⁶ are as defined above; by one of the following:

a) reducing the compound of formula (17) with a hydride in the presence of an acid;

b) hydrogenating the compound of formula (17) with a metal catalyst; or

c) transfer hydrogenating the compound of formula (17) with a metal catalyst; thereby making a compound of formula (16), or a salt thereof.

In some embodiments, the hydride is a borohydride. In some embodiments, the borohydride is selected from LiBH₄, NaBH₄, KBH₄, of NaBH₃CN. In some embodiments, the borohydride is NaBH₃CN. In some embodiments, the acid is a sulphonic acid. In some embodiments, the sulphonic acid is selected from: p-toluenesulphonic acid, methanesulphonic acid, and benzenesulphonic acid. In some embodiments, the sulphonic acid is p-toluenesulphonic acid. In some embodiments, the metal catalyst is Pd or Ni. In some embodiments, the metal catalyst is Pd—C. In some embodiments, the transfer hydrogenation further comprises a formate (e.g., sodium formate) as a hydrogen source.

In some embodiments, R⁴ is a C₅₋₁₂ aryl. In some embodiments, R⁴ is 4-(2-pyridyl)phenyl. In some embodiments, R⁵ is an amino acid side chain. In some embodiments, R⁵ is a C₁₋₁₀ alkyl. In some embodiments, R⁵ is tert-butyl. In some embodiments, R⁶ is a C₁₋₁₀ alkyl. In some embodiments, R⁶ is methyl. In some embodiments, the compound of formula (16) is:

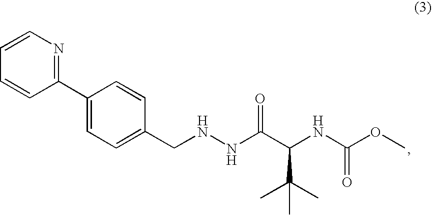

or a salt thereof.

A method of making a compound of formula (3):

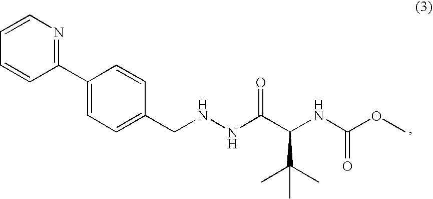

or a salt thereof, is provided herein, the method comprising reacting a compound of formula (18):

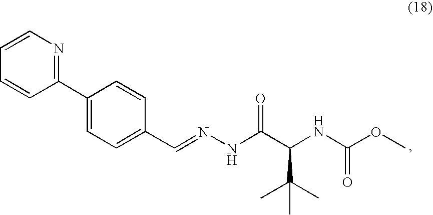

or a salt thereof, by one of the following:

a) reducing the compound of formula (18) with a hydride in the presence of an acid;

b) hydrogenating the compound of formula (18) with a metal catalyst; or

c) transfer hydrogenating the compound of formula (18) with a metal catalyst; thereby making a compound of formula (3), or a salt thereof.

Provided herein is a method of making a compound of formula (9):

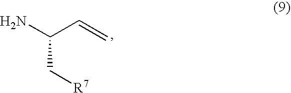

or a salt thereof, wherein:

R⁷ is a C₄₋₁₀ alkyl, C₅₋₁₂ cycloalkyl, C₅₋₁₂ heterocycloalkyl, C₅₋₁₂ aryl, C₅₋₁₂ heteroaryl, and an amino acid side chain, wherein the alkyl, cycloalkyl, heterocycloalkyl, aryl and heteroaryl can be substituted or unsubstituted;

the method comprising reacting a compound of formula (10) or (11):

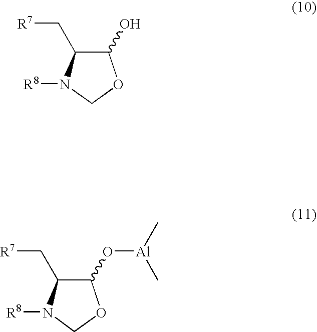

or a salt thereof, wherein:

R⁷ is as defined above; and

R⁸ is an amino protecting group;

with one of the following:

a) Ar₃P═CH₂, wherein Ar is an unsubstituted or substituted aryl or heteroaryl;

b) CH₂I₂—Zn—AlMe₃, followed by acid or base work-up; or

c) a compound of formula (14):

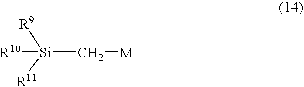

wherein:

R⁹, R¹⁰, and R¹¹ are independently a substituted or unsubstituted C₁₋₁₀ alkyl or C₅₋₁₂ aryl; and

M is Li, MgCl, MgBr, or MgI;

followed by treatment with a strong acid; thereby making a compound of formula (9) or a salt thereof.

In some embodiments, R⁷ is an amino acid side chain. In some embodiments, the amino acid side chain is selected from a side chain of histidine, phenylalanine, tryptophan, and tyrosine. In some embodiments, R⁷ is a C₅₋₁₂ aryl. In some embodiments, R⁷ is phenyl. In some embodiments, the amino protecting group is selected from alkyloxycarbonyl, triarylmethyl, tert-butyloxycarbonyl (Boc), or triphenylmethyl (Tr). In some embodiments, the amino protecting group is tert-butyloxycarbonyl. In some embodiments, Ar is selected from phenyl, anthracyl, and naphthyl. In some embodiments, Ar is phenyl.

In some embodiments, reaction a) is conducted under standard Wittig reaction conditions. In some embodiments, the strong acid is selected from BF₃.Et₂O, H₂SO₄, or HO₂CCF₃. In some embodiments, the compound of formula (9) is:

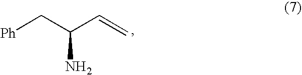

or a salt thereof.

Further provided herein is a method of making a compound of formula (7):

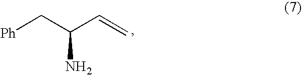

or a salt thereof, the method comprising reacting a compound of formula (12) or (13):

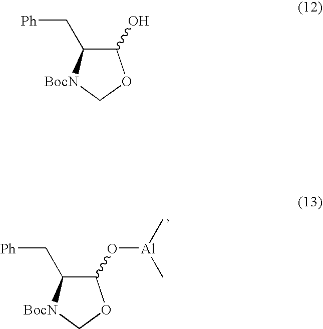

or a salt thereof, with one of the following:

a) Ar₃P═CH₂, wherein Ar is an unsubstituted or substituted aryl or heteroaryl;

b) CH₂I₂—Zn—AlMe₃, followed by acid or base work-up; or

c) a compound of formula (14):

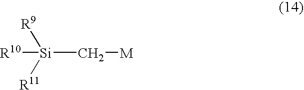

wherein:

R⁹, R¹⁰, and R¹¹ are independently a substituted or unsubstituted C₁₋₁₀ alkyl or C₅₋₁₂ aryl; and

M is Li, MgCl, MgBr, or MgI;

followed by treatment with a strong acid; thereby making a compound of formula (7) or a salt thereof.

Provided herein is a compound according to formula (16)

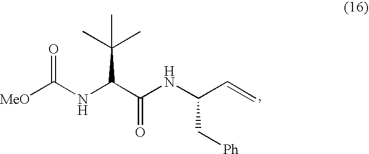

or a salt thereof. Also provided herein is a composition comprising a carrier and a compound according to formula (16). In some embodiments, the carrier is a pharmaceutically acceptable carrier.

Also provided herein is a compound according to formula (2):

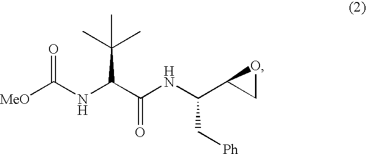

or a salt thereof. Further provided herein is a composition comprising a carrier and a compound according to formula (2). In some embodiments, the carrier is a pharmaceutically acceptable carrier.

The details of one or more embodiments of the invention are set forth in the accompanying drawings and the description below. Other features, objects, and advantages of the invention will be apparent from the description and drawings, and from the claims.

### DETAILED DESCRIPTION

### I. Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The term “alkyl,” by itself or as part of another substituent, means, unless otherwise stated, a straight or branched chain, which may be fully saturated, mono- or polyunsaturated, can include di- and multivalent radicals, and can have a number of carbon atoms optionally designated (i.e., C₁-C₈ means one to eight carbons). Examples of saturated hydrocarbon groups include, but are not limited to, groups such as methyl, ethyl, n-propyl, isopropyl, n-butyl, t-butyl, isobutyl, sec-butyl, n-pentyl, isopentyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotonyl, 2-isopentenyl, 2-(butadienyl), 2,4-pentadienyl, 3-(1,4-pentadienyl), ethynyl, 1- and 3-propynyl, 3-butynyl, and higher homologs and isomers.

The term “cycloalkyl”, by itself or in combination with other terms, represents, unless otherwise stated, cyclic versions of substituted or unsubstituted “alkyl”. Examples of cycloalkyl include, but are not limited to, cyclopentyl, cyclohexyl, 1-cyclohexenyl, 3-cyclohexenyl, cycloheptyl, and the like. The carbon atoms of the cyclic structures are optionally oxidized.

The term “heterocycloalkyl” as used herein refers to a cycloalkyl having a heteroatom. The heteroatom can occupy any position, including the position at which the heterocycle is attached to the remainder of the molecule. Examples of heterocycloalkyl include, but are not limited to, pyrrolidinyl, 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, dihydroimidazolyl, benzoimidazolyl, dihydrooxazolyl, and the like. The heteroatoms and carbon atoms of the cyclic structures are optionally oxidized or, in the case of N, quaternized.

The terms “halo” or “halogen,” by themselves or as part of another substituent, mean, unless otherwise stated, a fluorine, chlorine, bromine, or iodine atom.

The term “aryl” means, unless otherwise stated, a polyunsaturated, aromatic, hydrocarbon moiety which can be a single ring or multiple rings (e.g., from 1 to 3 rings) which are fused together or linked covalently. The term “heteroaryl” refers to aryl groups (or rings) that contain from one to four heteroatoms selected from N, O, and S, wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. “Aryl” and “heteroaryl” also encompass ring systems in which one or more non-aromatic ring systems are fused, or otherwise bound, to an aryl or heteroaryl system. Aryl-containing groups include, but are not limited to, phenyl, phenoxycarbonyl, benzoyl, benzyl, phenylpiperidinyl, phenylmorpholinyl, and dihydrobenzodioxyl (e.g., N,N-dihydrobenzodioxyl).

As used herein, “substituted” or “optionally substituted” refers to substitution by one or more substituents, in some embodiments one, two, three, or four substituents. In some embodiments, two substituents may join to form a cyclic or heterocyclic ring containing 3-7 atoms. Non-limiting examples of substituents include C₁₋₁₀ alkyl; OR; halo; NR′R″; NO₂; CN; SR′; SO₂; COOR′; C₅₋₁₂ cycloalkyl; C₅₋₁₂ heterocycloalkyl; C₅₋₁₂ aryl; and C₅₋₁₂ heteroaryl; wherein the alkyl, cycloalkyl, heterocycloalkyl, aryl, or heteroaryl may be substituted or unsubstituted, wherein each R′ and R″ is independently H or C₁₋₁₀ substituted or unsubstituted alkyl. In some embodiments, a substituent is selected from C₁₋₆ alkyl, halo, and OR′.

As used herein, the term “heteroatom” includes oxygen (O), nitrogen (N), sulfur and (S).

As used herein, the term “amino acid” includes natural and unnatural amino acids. In some embodiments, an amino acid may be substituted. As used herein, the term “natural” or “naturally occurring” amino acid refers to one of the twenty most common occurring amino acids. Non-limiting examples of unnatural amino acids include: L-1-Naphthylalanine; L-2-Naphthylalanine; L-2-Pyridylalanine; L-3-Nitrotyrosine; L-4-carboxyphenylalanine; L-4-Pyridylalanine; L-4-tert-Butylphenylalanine; L-α-Aminobutyic acid; Aminoisobutyric acid; L-β-homoaspartic acid; L-βp-homoserine; L-β-homotryptophan; L-β-homotyrosine; L-Biphenylalanine; D-Biphenylalanine; L-4-Benzoylphenylalanine; L-Cyclohexylalanine; L-Citrulline; L-Diphenylalanine; L-3,4-Dihydroxyphenylalanine; L-3,4-Dehydroproline; L-3,4-Dimethoxyphenylalanine; L-3-Methoxyhenylalanine; L-4-Trifluoromethylphenylalanine; L-4-Cyanophenylalanine; L-4-Fluorophenylalanine; L-4-Aminophenylalanine; L-4-Nitrophenylalanine; L-Homophenylalanine; L-Homoserine; L-Homotyrosine; L-O-methylhomotyrosine; N-Methyl-L-alanine; L-Ornithine; L-3-Hydroxyproline; L-Penicillamine; L-Pipecolic acid; L-3-Benzothienylalanine; L-1,2,3,4-Tetrahydroisoquinoline-3-carboxylic acid; L-tert-Leucine; L-5-Hydroxytryptophan; L-2,6-Dimethyltyrosine; L-3-Chlorotyrosine; L-3-Iodotyrosine; and L-3-Chloro-O-benzyltyrosine.

### II. Method of Making

Provided herein is an improved process for preparing Atazanavir, a compound of formula (1):

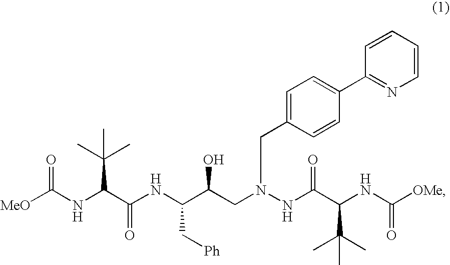

or a salt thereof.

In some embodiments, the process includes reacting a compound of formula (2):

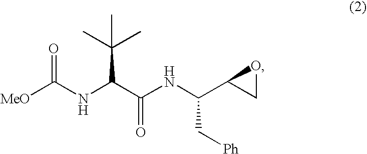

or a salt thereof, with a compound of formula (3):

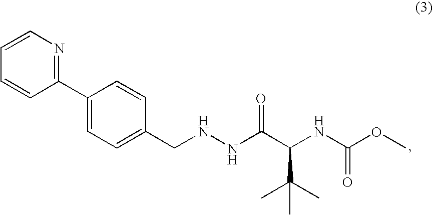

or a salt thereof. This reaction takes place in the presence of a Lewis acid catalyst in an inert solvent at a temperature from about 0 to about 140° C.

A Lewis acid catalyst can include a metal triflate, such as Li(OTf), Sn(OTf)₂, Cu(OTf)₂, Bi(OTf)₃, Ca(OTf)₂, Al(OTf)₃, Sm(OTf)₃, Yb(OTf)₃, and Sc(OTf)₃; a metal halide, such as CeCl₃, WCl₆, ZrCl₄, RuCl₃, SbCl₃, CoCl₂, CdCl₂, TaCl₅, InCl₃, BiCl₃, VCl₃, SnCl₄, ZnCl₂, ZrCl₄, InBr₃, MgBr₂, LiBr, SmI₂, and SmCl₃; a metal perchlorate, such as LiClO₄, NaClO₄, Zn(ClO₄)₂, and Cu(ClO₄)₂; a metal tetrafluoroborate, such as LiBF₄, Zn(BF₄)₂, and Cu(BF₄)₂; a fluoroalkyl alcohol, such as hexafluoro-2-propanol; and a heterogeneous catalyst, such as alumina, phosphomolybdic acid-Al₂O₃, Zn(ClO₄)₂—Al₂O₃, silica gel (SiO₂), heteropolyacids, zirconium sulfophenyl phosphonates, zeolites, clays, mesoporous aluminosilicates, K₅CoW₁₂O₄₀.3H₂O, (NH₄)₈[CeW₁₀O₃₆].20H₂O, Montmorillonite K-10; SBA-15, and Amberlist-15. In some embodiments, the Lewis acid is a heterogeneous catalyst. In some embodiments, the Lewis acid is SiO₂.

An inert solvent can include water; an alcohol, such as methanol, ethanol, n-propanol, isopropanol, n-butanol, isobutanol, tert-butanol, and ethylene glycol; an ether, such as diethyl ether, diisopropyl ether, tetrahydrofuran, and dioxane; an amide, such as dimethylformamide and dimethylacetamide; an ester, such as ethyl acetate, methyl acetate, and ethyl formate; a chlorinated hydrocarbon, such as dichloromethane, chloroform, and dichloroethane; and a hydrocarbon, such as hexane, heptane, benzene, and toluene. In some embodiments, the solvent is a chlorinated hydrocarbon. In some embodiments, the solvent is dichloromethane.

The reaction temperature can be any value or range between, and including, about 0 to about 140° C. For example, the temperature can be from about 0° C. to about 120° C.; from about 0° C. to about 100° C.; from about 0° C. to about 90° C.; from about 0° C. to about 75° C.; from about 0° C. to about 50° C.; from about 0° C. to about 20° C.; from about 5° C. to about 140° C.; from about 10° C. to about 140° C.; from about 20° C. to about 140° C.; from about 25° C. to about 140° C.; from about 35° C. to about 140° C.; from about 40° C. to about 140° C.; from about 50° C. to about 140° C.; from about 70° C. to about 140° C.; from about 85° C. to about 140° C.; from about 90° C. to about 140° C.; from about 100° C. to about 140° C.; from about 120° C. to about 140° C.; from about 20° C. to about 120° C.; from about 25° C. to about 100° C.; from about 30° C. to about 90° C.; from about 40° C. to about 85° C.; and from about 50° C. to about 75° C.

Also provided herein is a process for preparing a compound of formula (4):

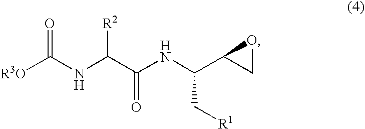

or a salt thereof, wherein:
R¹ is selected from C₄₋₁₀ alkyl, C₅₋₁₂ cycloalkyl, C₅₋₁₂ heterocycloalkyl, C₅₋₁₂ aryl, C₅₋₁₂ heteroaryl, and an amino acid side chain, wherein the alkyl, cycloalkyl, heterocycloalkyl, aryl and heteroaryl can be substituted or unsubstituted; and
R² is selected from H, C₁₋₁₀ substituted or unsubstituted alkyl, and an amino acid side chain; and
R³ is selected from H and C₁₋₁₀ substituted or unsubstituted alkyl.
   The process includes reacting a compound of formula (5):

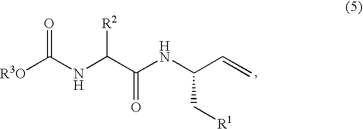

or a salt thereof, wherein R¹, R², and R³ are as defined above.

In some embodiments, R¹ is an amino acid side chain. Non-limiting examples of an amino acid side chain, include a side chain of histidine, phenylalanine, tryptophan, and tyrosine. In some embodiments, R¹ is a C₅₋₁₂ aryl. In some embodiments, R¹ is phenyl. In some embodiments, R² is an amino acid side chain. In some embodiments, R² is a C₁₋₁₀ alkyl. In some embodiments, R² is tert-butyl. In some embodiments, R³ is a C₁₋₁₀ alkyl. In some embodiments, R³ is methyl. In some embodiments, the compound of formula (4) is a compound of formula (2):

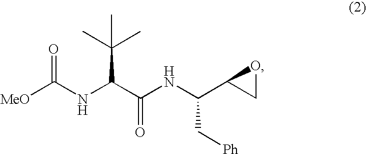

or a salt thereof. In some embodiments, a compound of formula (5) is a compound of formula (6):

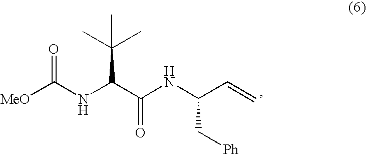

or a salt thereof.

In some embodiments, the reaction of a compound of formula (5) occurs through an epoxidation reaction with one or more of molecular oxygen (e.g., O₂ or air), hydrogen peroxide, or an organic peroxide. In some embodiments, the reaction utilizes a metal catalyst. In some embodiments, the reaction occurs with hydrogen peroxide in the presence of a metal catalyst. In some embodiments, a metal catalyst can be selected from a transition metal oxide solid acid (TMO); tungstic acid or its derivatives; vanadium complexes; molybdenum complexes; titanium complexes; and cobalt complexes. In some embodiments, the reaction occurs at a temperature ranging from −40 to 100° C.

In some embodiments, the reaction of a compound of formula (5) occurs through an epoxidation reaction with a peroxy acid, or a salt thereof. Non-limiting examples of a peroxy acid, or a salt thereof, include perbenzoic acid, performic acid, peracetic acid, monoperoxyphthalic acid, pertungstic acid, m-chloroperbenzoic acid, or magnesium bis-monoperoxyphthalate hexahydrate (MMPP). In some embodiments, the peroxy acid, or a salt thereof, is MMPP. In some embodiments, the reaction of a compound of formula (5) occurs with a peroxy acid in the presence of a solvent, such as such as an alcohol, for example, methanol, ethanol, and iso-propanol); an ether, for example diethyl ether or tetrahydrofuran; or a chlorinated hydrocarbons, for example, chloroform or dichloromethane. In some embodiments, the solvent is methanol. In some embodiments, the reaction occurs at a temperature ranging from −40 to 100° C.

In some embodiments, the reaction occurs with a peroxy acid, or a salt thereof, in a solvent such as an alcohol or a chlorinated hydrocarbon and at a temperature ranging from −20 to 80° C. In some embodiments, the peroxy acid, or a salt thereof, is selected from perbenzoic acid, monoperoxyphthalic acid, m-chloroperbenzoic acid, or magnesium monoperoxyphthalate hexahydrate (MMPP), and performed in a solvents, such as methanol, ethanol, iso-propanol, chloroform, or dichloromethane at temperatures of from about 0 to 60° C. In some embodiments, the peroxy acid, or salt thereof is MMPP, the solvent is methanol, and the reaction is performed at a temperature from 10 to 50° C.

In some embodiments, a compound of formula (6) is reacted with a peroxy acid, or a salt thereof, to produce a compound of formula (2). In some embodiments, the peroxy acid is MMPP. In some embodiments, the reaction is performed in methanol. In some embodiments, a compound of formula (2) can be prepared by a stereoselective process. See, e.g., Example 4.

A method for preparing a compound of formula (6) is also provided herein. The compound can be prepared by coupling a compound of formula (7):

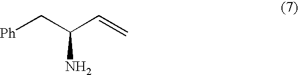

or a salt thereof, with a compound of formula (8):

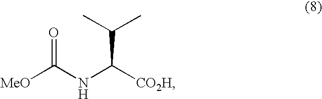

or a salt thereof. The compounds can be coupled in situ under standard peptide formation conditions.

Provided herein is a method for preparing a compound of formula (9):

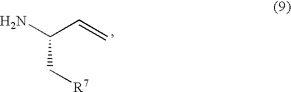

or a salt thereof, wherein:
R⁷ is a C₄₋₁₀ alkyl, C₅₋₁₂ cycloalkyl, C₅₋₁₂ heterocycloalkyl, C₅₋₁₂ aryl, C₅₋₁₂ heteroaryl, and an amino acid side chain, wherein the alkyl, cycloalkyl, heterocycloalkyl, aryl and heteroaryl can be substituted or unsubstituted.
   In some embodiments, R⁷ is an amino acid side chain. Non-limiting examples of an amino acid side chain, include a side chain of histidine, phenylalanine, tryptophan, and tyrosine. In some embodiments, R⁷ is a C₅₋₁₂ aryl. In some embodiments, R⁷ is phenyl. In some embodiments, a compound of formula (9) is a compound of formula (7).

A compound of formula (9), as described above, can be prepared by reacting a lactol of formula (10):

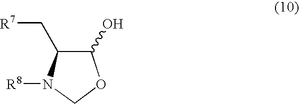

or a salt thereof, or an aluminoxy acetal of formula (11):

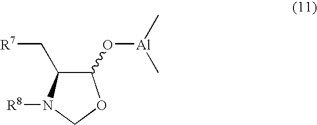

or a salt thereof; wherein R⁷ is as described above, and R⁸ is an amino protecting group. Non-limiting examples of amino protecting groups include alkyloxycarbonyl, triarylmethyl, tert-butyloxycarbonyl (Boc), or triphenylmethyl (Tr). In some embodiments, R⁸ is tert-butyloxycarbonyl (Boc). In some embodiments, the compound of formula (10) is a compound of formula (12):

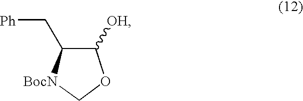

or a salt thereof. In some embodiments, a compound of formula (11) is a compound of formula (13):

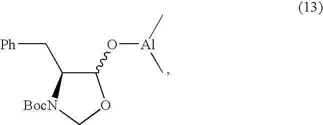

or a salt thereof.

In some embodiments, a compound of formula (9) can be prepared by the reaction of a compound of formula (10) or (11) with an ylide having a formula Ar₃P═CH₂, wherein Ar is a substituted or unsubstituted C₅₋₁₂ aryl group. In some embodiments, the aryl group is selected from phenyl, anthracyl, and naphthyl. In some embodiments, Ar is phenyl. The reaction of a compound of formula (10) or (11) with the ylide can occur under standard Witting reaction conditions. In some embodiments, the reaction is followed by acid/base work-up.

In some embodiments, a compound of formula (9) can be prepared by the reaction a compound of formula (10) or (11) can be reacted with CH₂I₂—Zn—AlMe₃, followed by acid/base work-up.

In some embodiments, a compound of formula (9) can be prepared by the reaction a compound of formula (10) or (11) can be reacted with a compound of formula (14):

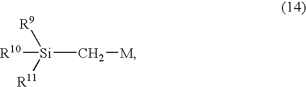

or a salt thereof, wherein:
R⁹, R¹⁰, and R¹¹ are independently a substituted or unsubstituted C₁₋₁₀ alkyl or C₅₋₁₂ aryl; and
M is Li, MgCl, MgBr, or MgI.
   The reaction is followed by treatment with a strong acid (e.g., BF₃.Et₂O, H₂SO₄, or HO₂CCF₃).

In some embodiments, a compound of formula (9) is prepared by the reactions, as described above, using a compound of formula (11). In some embodiments, a compound of formula (7) is prepared using a compound of formula (12) or (13). In some embodiments, a compound of formula (7) is prepared using a compound of formula (13). In some embodiments, a compound of formula (7) is prepared by reacting a compound of formula (12) or (13) with Ar₃Ph=CH₂. In some embodiments, a compound of formula (7) is prepared by reacting a compound of formula (12) or (13) with Ph₃Ph=CH₂. In some embodiments, the acid/base work-up is performed with hydrochloric acid. See, e.g., Example 2.

A method of preparing a compound of formula (12) or (13) is provided herein. The method includes reducing a lactone of formula (15):

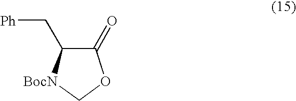

or a salt thereof, with either (a) a borohydride or (b) an aluminohydride. A borohydride can include MBH₄ or MHBR₃; wherein M is selected from Li, Na, K, Bu₄N, Zn or Ca; and R is a C₁₋₁₀ alkyl. In some embodiments, the borohydride is selected from LiBH₄, NaBH₄, or KBH₄. In some embodiments, the borohydride is LiBH₄ or NaBH₄. An aluminohydride can include HAlR₂, MHAl(OR)₃, and MH₂Al(OR)₂; wherein M is selected from Li, Na, K, Bu₄N, Zn or Ca; and R is a C₁₋₁₀ alkyl. In some embodiments, the aluminohydride is selected from HAl(Bu-i)₂, LiHAl(O-Bu-t)₃, LiHAl(OMe)₃, LiHAl(OEt)₃, and NaH₂Al(OCH₂CH₂OCH₃)₂. In some embodiments, the aluminohydride is HAl(Bu-i)₂ or LiHAl(O-Bu-t)₃.

Further provided herein is a method of preparing a compound of formula (16):

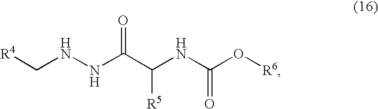

or a salt thereof, wherein:
R⁴ is selected from C₄₋₁₀ alkyl, C₅₋₁₂ cycloalkyl, C₅₋₁₂ heterocycloalkyl, C₅₋₁₂ aryl, C₅₋₁₂ heteroaryl, and an amino acid side chain, wherein the alkyl, cycloalkyl, heterocycloalkyl, aryl and heteroaryl can be substituted or unsubstituted; and
R⁵ is selected from H, C₁₋₁₀ substituted or unsubstituted alkyl, and an amino acid side chain; and
R⁶ is selected from H and C₁₋₁₀ substituted or unsubstituted alkyl.
   The process involves reducing or hydrogenating a compound a compound of formula (17):

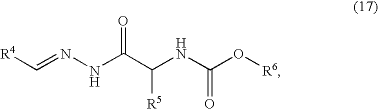

or a salt thereof, wherein R⁴, R⁵, and R⁶ are as defined above.

In some embodiments, R⁴ is a C₅₋₁₂ aryl. In some embodiments, R⁴ is 4-(2-pyridyl)phenyl. In some embodiments, R⁵ is an amino acid side chain. In some embodiments, R⁵ is a C₁₋₁₀ alkyl. In some embodiments, R⁵ is tert-butyl. In some embodiments, R⁶ is a C₁₋₁₀ alkyl. In some embodiments, R⁶ is methyl. In some embodiments, a compound of formula (16) is a compound of formula (3):

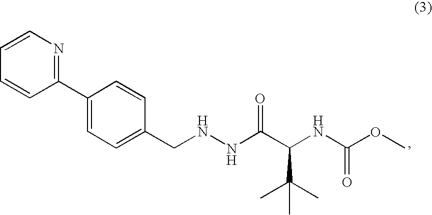

or a salt thereof. In some embodiments, a compound of formula (17) is a compound of formula (18):

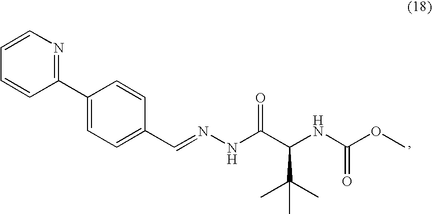

or a salt thereof.

In some embodiments, a compound of formula (16) is prepared by reducing a compound of formula (17) with a hydride in the presence of an acid. In some embodiments, the hydride is a borohydride (e.g., LiBH₄, NaBH₄, KBH₄, of NaBH₃CN). In some embodiments, the borohydride is NaBH₃CN. In some embodiments, the acid is a sulphonic acid (e.g., p-toluenesulphonic acid, methanesulphonic acid, and benzenesulphonic acid). In some embodiments, the acid is p-toluenesulphonic acid.

In some embodiments, a compound of formula (16) is prepared by hydrogenation of a compound of formula (17) in the presence of a metal catalyst. In some embodiments, the metal catalyst is a Pt, Pd, or Ni catalyst. In some embodiments, the metal catalyst is a Pd or Ni catalyst. In some embodiments, the metal catalyst is Pd—C.

In some embodiments, a compound of formula (16) is prepared by transfer hydrogenation of a compound of formula (17) in the presence of a metal catalyst. In some embodiments, the metal catalyst is a Pt, Pd, or Ni catalyst. In some embodiments, the metal catalyst is a Pd or Ni catalyst. In some embodiments, the metal catalyst is Pd—C. In some embodiments, formate (e.g., sodium formate, potassium formate, and ammonium formate) is used in the reaction as a hydrogen source.

In some embodiments, a compound of formula (3) is prepared by reaction of a compound of formula (18) in the presence of Pd—C and sodium formate. In some embodiments, the reaction is performed in ethanol. See, e.g., Example 7.

A method of preparing a compound of formula (18) is also provided herein. The reaction comprises the coupling of a compound of formula (19):

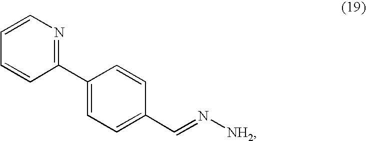

or a salt thereof, with a compound of formula (20):

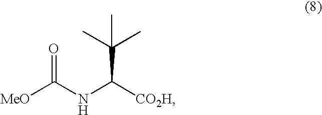

or a salt thereof. The compounds can be coupled in situ under standard peptide formation conditions.

A compound of formula (19), or a salt thereof, can be prepared by the reaction of a compound of formula (20):

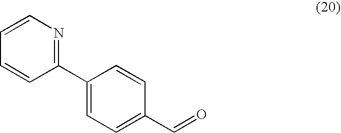

through condensation with hydrazine hydrate in a lower alcohol (e.g., methanol, ethanol, or isopropanol.) In some embodiments, the reaction is conducted in methanol.

As described above, a compound of formula (1):

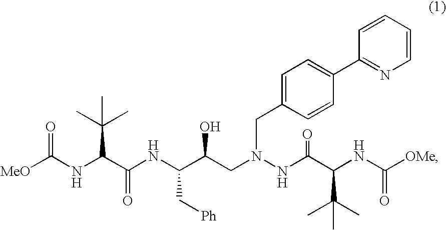

or a salt thereof, can be synthesized using the following methods:

a) reacting a compound of formula (12) of (13):

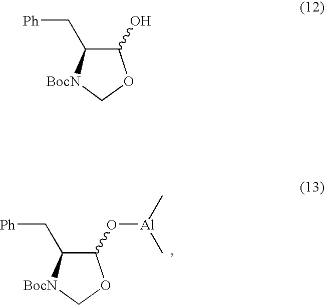

or a salt thereof, with Ar₃P═CH₂, wherein Ar is a substituted or unsubstituted C₄₋₁₂ aryl, followed by acid or base work-up, to produce a compound of formula (7):

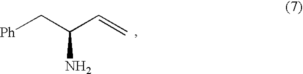

or a salt thereof;

b) coupling the compound of formula (7) with a compound of formula (8):

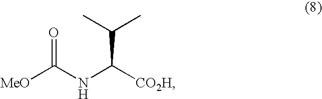

or a salt thereof, to produce a compound of formula (6):

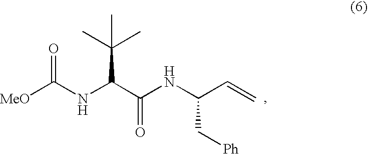

or a salt thereof;

c) reacting the compound of formula (6) with a peroxy acid, or a salt thereof, to produce a compound of formula (2):

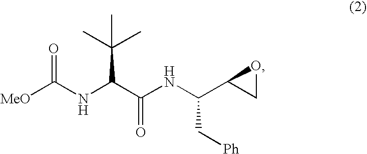

or a salt thereof;

d) reacting 4-(2-pyridyl)benzaldehyde with hydrazine hydrate in a lower alcohol to produce a compound of formula (19):

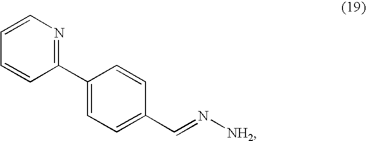

or a salt thereof;

e) coupling the compound of formula (19) with a compound of formula (8):

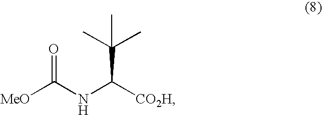

or a salt thereof, to produce a compound of formula (18):

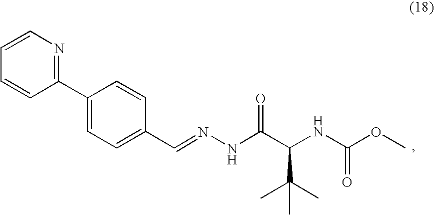

or a salt thereof;

f) hydrogenating the compound of formula (18) with a metal catalyst to produce a compound of formula (3):

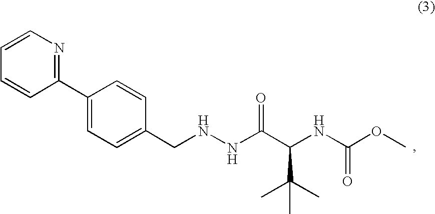

or a salt thereof; and

g) reacting the compound of formula (2), or a salt thereof, with the compound of formula (3), or a salt thereof, in the presence of a Lewis acid catalyst to make the compound of formula (1), or a salt thereof.

In some embodiments a compound of formula (1):

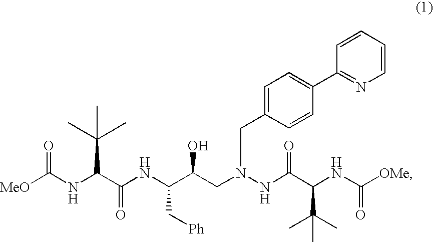

or a salt thereof, is prepared by:

a) reacting a compound of formula (13):

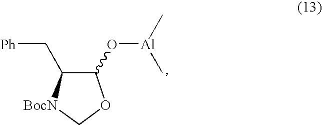

or a salt thereof, with Ph₃P═CH₂ with hydrochloric acid, to produce a compound of formula (7):

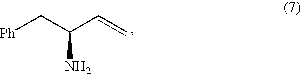

or a salt thereof;

b) coupling the compound of formula (7) with a compound of formula (8):

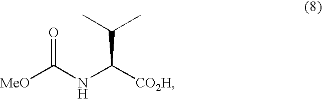

or a salt thereof, to produce a compound of formula (16):

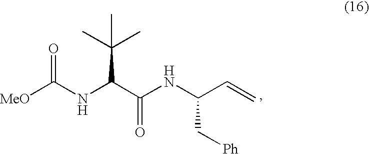

or a salt thereof;

c) reacting the compound of formula (16) with magnesium bis-monoperoxyphthalate hexahydrate (MMPP), to produce a compound of formula (2):

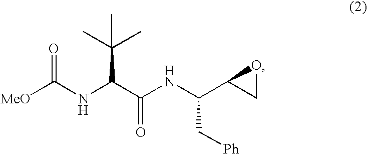

or a salt thereof;

d) reacting 4-(2-pyridyl)benzaldehyde with hydrazine hydrate in a lower alcohol to produce a compound of formula (19):

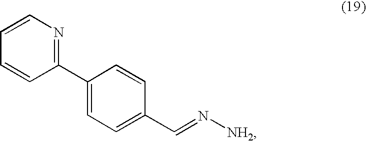

or a salt thereof;

e) coupling the compound of formula (g) with a compound of formula (h):

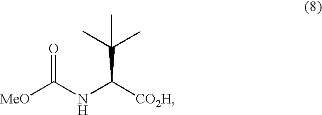

or a salt thereof, to produce a compound of formula (18):

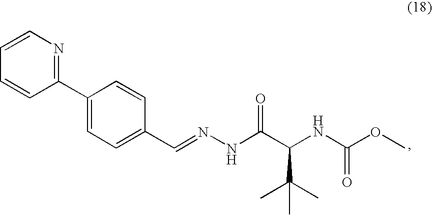

or a salt thereof;

f) hydrogenating the compound of formula (18) with sodium formate and Pd—C to produce a compound of formula (3):

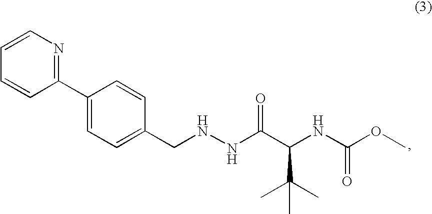

or a salt thereof; and

g) reacting the compound of formula (2), or a salt thereof, with the compound of formula (3), or a salt thereof, in the presence of SiO₂ to make the compound of formula (1), or a salt thereof. See, e.g., Scheme 1:

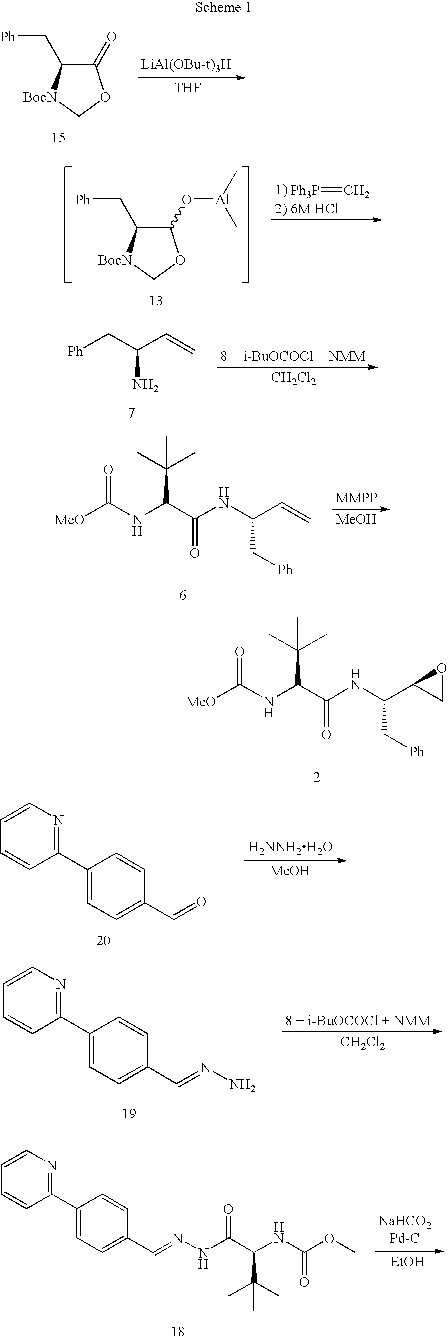

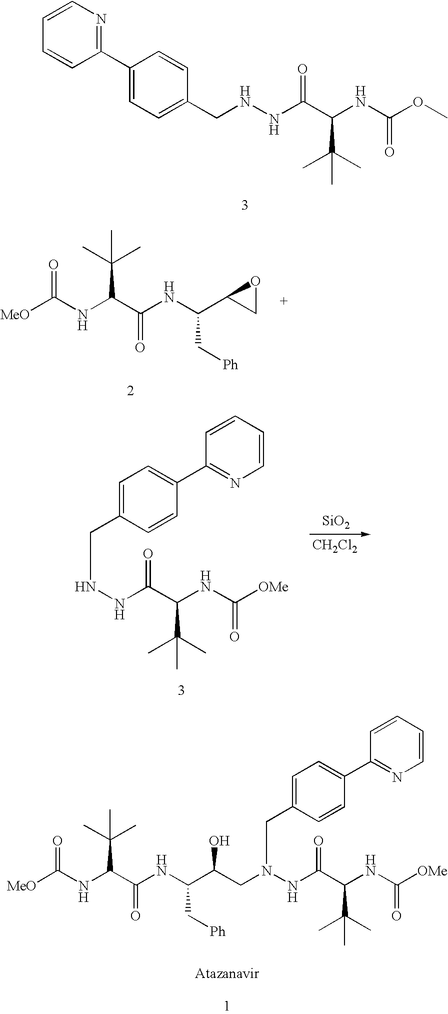

In some methods, an improvement to the known route to a compound of formula (1) might be useful, in such a case, a reaction as described below could be followed.

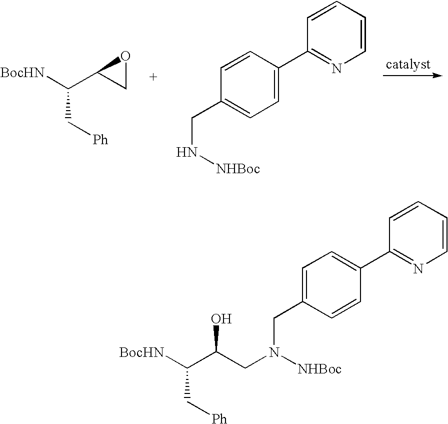

The methods and compounds described herein may be useful in the synthesis of other similar types of compounds. For example, in the preparation of the compounds or intermediates disclosed in WO 2008/011117; WO 2008/011116; WO 2007/002173; WO 2001/089282; Bioorganic & Medicinal Chemistry Letters, 15(15): 3560-3564, 2005; and Journal of Medicinal Chemistry, 50(18): 4316-4328, 2007.

### III. Novel Compounds and Compositions

Also provided herein are a compound of formula (2):

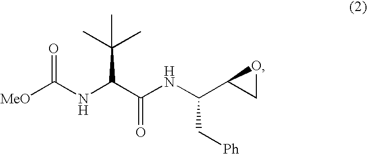

or a salt thereof. Further provided herein is a compound of formula (6):

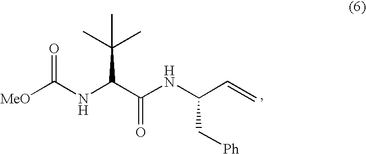

or a salt thereof.

The compounds described above can also be formulated as a composition comprising one or more of compounds (2) and (6) and a carrier. In some embodiments, a carrier is a pharmaceutical carrier.

The compounds of described herein may be administered in the form of a composition (e.g., a pharmaceutical composition), in combination with an acceptable carrier (e.g., a pharmaceutically acceptable carrier). The active ingredient in such formulations may comprise from 0.1 to 99.99 weight percent. “Pharmaceutically acceptable carrier” means any carrier, diluent or excipient which is compatible with the other ingredients of the formulation and not deleterious to the recipient.

The active agent may be administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington: The Science and Practice of Pharmacy, 20th Edition (2003), Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorobutanol. The composition for parenteral administration may take the form of an aqueous or non-aqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The specific dose of a compound, as described herein, required to obtain therapeutic benefit in the methods of treatment described herein will, of course, be determined by the particular circumstances of the individual patient including the size, weight, age and sex of the patient, the nature and stage of the disease being treated, the aggressiveness of the disease disorder, and the route of administration of the compound.

The compounds disclosed herein, any of the embodiments thereof, may take the form of salts. The term “salts” embraces addition salts of free acids or free bases which are compounds described herein. The term “pharmaceutically-acceptable salt” refers to salts which possess toxicity profiles within a range that affords utility in pharmaceutical applications. Pharmaceutically unacceptable salts may nonetheless possess properties such as high crystallinity, which may render them useful, for example in processes of synthesis, purification or formulation of compounds described herein. In general the useful properties of the compounds described herein do not depend critically on whether the compound is or is not in a salt form, so unless clearly indicated otherwise (such as specifying that the compound should be in “free base” or “free acid” form), reference in the specification to the compounds described herein should be understood as encompassing salt forms of the compound, whether or not this is explicitly stated.

Suitable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of additional acid addition salts include, for example, perchlorates and tetrafluoroborates.

Suitable base addition salts include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of additional base addition salts include lithium salts and cyanate salts.

All of these salts may be prepared by conventional means from the corresponding compound, as described herein, by reacting, for example, the appropriate acid or base with the compound. Preferably the salts are in crystalline form, and preferably prepared by crystallization of the salt from a suitable solvent. The person skilled in the art will know how to prepare and select suitable salt forms for example, as described in Handbook of Pharmaceutical Salts: Properties, Selection, and Use By P. H. Stahl and C. G. Wermuth (Wiley-VCH 2002).

### EXAMPLES

### Example 1

### (4S)—N-Boc-4-phenylmethyloxazolidin-5-one (15)

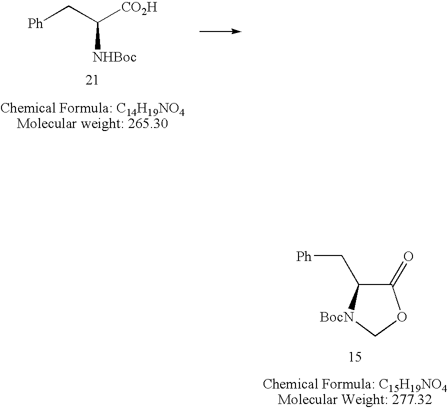

A mixture of N-Boc-L-phenylalanine (50.00 g, 188 mmol), paraformaldehyde (15.00 g, 470 mmol) and pyridinium p-toluenesulfonate (2.38 g, 9.4 mmol) in toluene (400 mL) was refluxed for 30 min. After cooling to room temperature, the mixture was washed with a saturated NaHCO₃ solution (200 mL) and brine (200 mL), dried (MgSO₄), and concentrated under reduced pressure to give the product (47.90 g, 92%).

### Example 2

### (S)-3-Amino-4-phenyl-1-butene hydrochloride (7)

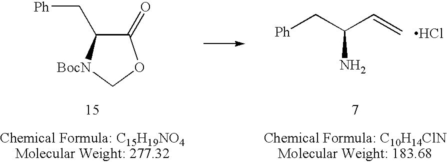

To a solution of compound 15 (8.32 g, 30 mmol) in THF (50 mL) was added a solution of LiHAl(OBu-t)₃ in THF (1M, 33 mL, 33 mmol) while keeping the temperature below −10° C. After addition, the solution was allowed to warm to 0° C. and stirred for 4 h at 0° C. to obtain an aluminoxy acetal (13) solution.

In another flask, to a suspension of methyltriphenylphosphonium bromide (23.58 g, 66 mmol) in THF (100 mL) was added potassium tert-butoxide (7.29 g, 65 mmol) at 0° C. After stirring for 1 h at 0° C., the aluminoxy acetal solution prepared above was added via cannula transfer. After stirring for 30 min at 0° C., the mixture was allowed to warm to 55° C. and stirred for 18 h at 55° C. After cooling to room temperature, the reaction was quenched with 6M HCl (150 mL) and the mixture was stirred for 3 h at 55° C. Most of the THF was then removed under reduced pressure. The residue was then extracted with EtOAc (2×50 mL). The acidic aqueous solution was adjusted to pH 10-12 with 25% NaOH solution, and extracted with EtOAc (2×50 mL). The extract was washed with brine (50 mL), dried (MgSO₄), and concentrated under reduced pressure. The residue was dissolved in CH₂Cl₂ (100 mL), acidified with 12 M HCl (2.5 mL), dried (MgSO₄), and concentrated under reduced pressure to give the product (3.51 g, 63.7%) as pale yellow solid.

### Example 3

### (3S)-3-{[N-(Methoxycarbonyl)-L-tert-leucinyl]amino}-4-phenyl-1-butene (6)

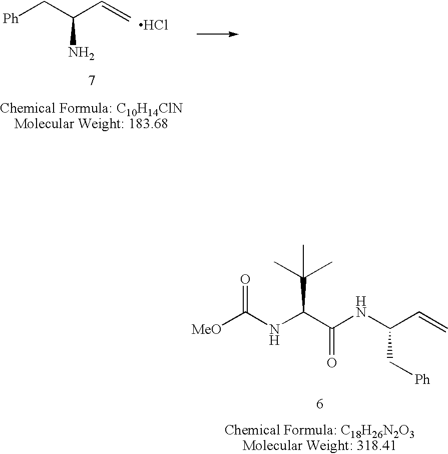

To a solution of N-methoxycarbonyl-L-tert-leucine (9.90 g, 52 mmol) and N-methylmorpholine(11.90 mL, 108 mmol) in CH₂Cl₂ (150 mL) was added dropwise isobutyl chloroformate (6.20 mL, 48 mmol) while keeping the temperature below −25° C. After stirring for 30 min at −25 to −22° C., (S)-3-Amino-4-phenyl-1-butene hydrochloride (7) (7.95 g, 43.3 mmol) as formed, the cooling bath was removed, and the mixture was stirred for another 2 h at room temperature. The mixture was washed with 1 M HCl (50 mL), water (50 mL), a saturated NaHCO₃ solution (50 mL) and brine (50 mL), dried (MgSO₄), and concentrated under reduced pressure. The residue was crystallized from hexane to give the product (11.17 g, 81%) as colorless needles.

### Example 4

### (2R,3S)-1,2-Epoxy-3-{[N-(methoxycarbonyl)-L-tert-leucinyl]amino}-4-phenylbutane (2)

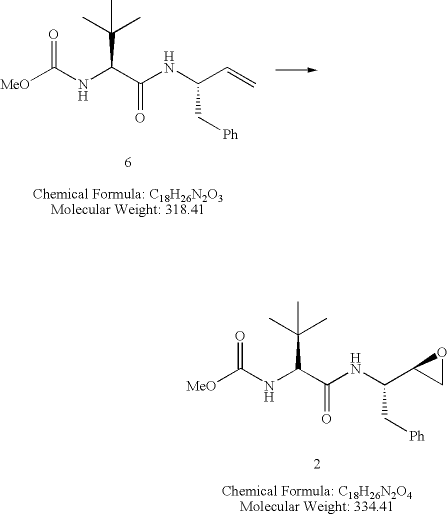

To a solution of (3S)-3-{[N-(Methoxycarbonyl)-L-tert-leucinyl]amino}-4-phenyl-1-butene (6) (3.18 g, 10 mmol) in MeOH (20 mL) was added magnesium bis(monoperoxyphthalate) hexahydrate (80%, 3.71 g, 6 mmol) at room temperature. After stirring for 32 h at room temperature, the mixture was diluted with water (100 mL), and extracted with EtOAc (100 mL). The extract was washed with water (100 mL), a saturated NaHCO₃ solution (50 mL) and brine (50 mL), dried (MgSO₄), and concentrated under reduced pressure. The residue was crystallized from EtOAc-hexane to give the product (2.94 g, 88%) as colorless crystals.

### Example 5

### N-[4-(2-pyridyl)phenylmethylidene]hydrazine (19)

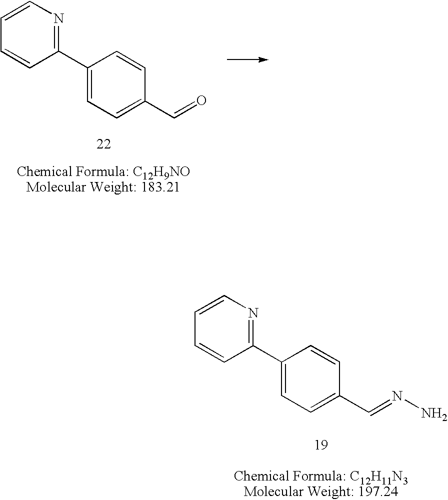

To a solution of hydrazine hydrate (98%, 7.50 mL, 150 mmol) in MeOH (30 mL) was added dropwise a solution of 4-(2-pyridyl)benzaldehyde (9.16 g, 50 mmol) in MeOH (30 mL) at room temperature. After stirring for 2 h at room temperature, volatile matters were removed under reduced pressure.

The residue was crystallized from EtOAc-hexane to give the product (9.27 g, 94%) as colorless crystals.

### Example 6

### N—[N-(Methoxycarbonyl)-L-tert-leucinyl]-N′-[4-(2-pyridyl)phenylmethylidene]hydrazine (18)

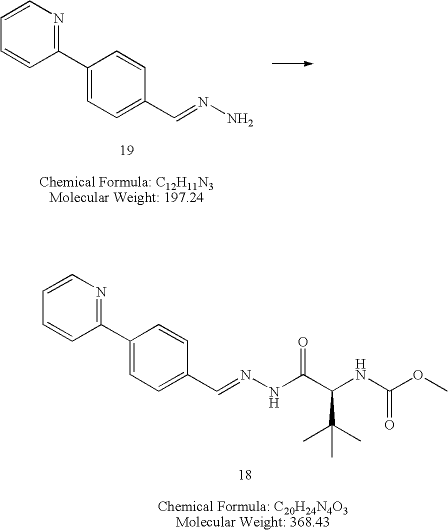

To a solution of N-methoxycarbonyl-L-tert-leucine (9.46 g, 50 mmol) and N-methylmorpholine (6.60 mL, 60 mmol) in CH₂Cl₂ (150 mL) was added dropwise isobutyl chloroformate (6.20 mL, 48 mmol) while keeping the temperature below −25° C. After stirring for 50 min at −25 to −30° C., N-[4-(2-pyridyl)phenylmethylidene]hydrazine (19) (8.88 g, 45 mmol) was added. After stirring for 30 min at −25° C., the cooling bath was removed, and the mixture was stirred for another 2 h at room temperature. The mixture was washed with water (2×100 mL), a saturated NaHCO₃ solution (50 mL) and brine (50 mL), dried (MgSO₄), and concentrated under reduced pressure. The residue was crystallized from EtOAc-hexane to give the product (14.10 g, 85%) as colorless crystals.

### Example 7

### N—[N-(Methoxycarbonyl)-L-tert-leucinyl]-N′-[4-(2-pyridyl)phenylmethyl]hydrazine (3)

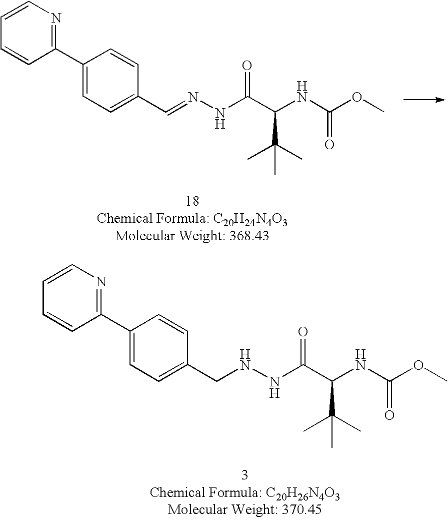

To a suspension of N—[N-(Methoxycarbonyl)-L-tert-leucinyl]-N′-[4-(2-pyridyl)phenylmethylidene]hydrazine (18) (14.74 g, 40 mmol) and 10% Pd—C (1.00 g) in EtOH (80 mL) was added a solution of sodium formate (5.44 g, 80 mmol) in water (8 mL). After stirring for 1 h at 60° C., the mixture was filtered through a pad of celite and washed with EtOAc. The combined filtrate and washings were concentrated under reduced pressure. The residue was partitioned between EtOAc (200 mL) and water (50 mL). The organic layer was separated, dried (MgSO₄), and concentrated under reduced pressure. The residue was crystallized from EtOAc-hexane to give the product (12.30 g, 83%) as colorless crystals.

### Example 8

### Atazanavir (1)

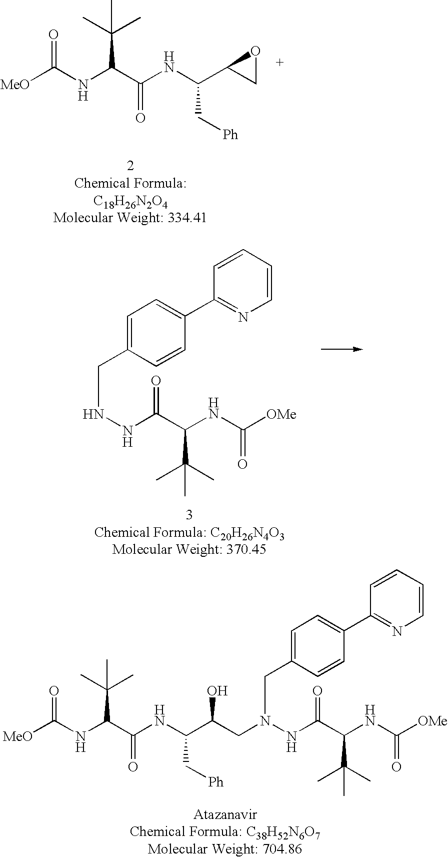

A suspension of (2R,3S)-1,2-Epoxy-3-{[N-(methoxycarbonyl)-L-tert-leucinyl]amino}-4-phenylbutane (2) (334 mg, 1 mmol), N—[N-(Methoxycarbonyl)-L-tert-leucinyl]-N′-[4-(2-pyridyl)phenylmethyl]hydrazine (3) (370 mg, 1 mmol) and silica gel (1.0 g) in CH₂Cl₂ (3 mL) was stirred for 64 h. The mixture was diluted with EtOAc (10 mL), filtered and washed with a mixture of EtOAc-CH₂Cl₂ (1:1). The filtrate was concentrated under reduced pressure. The residue was crystallized from EtOAc-hexane to give the product (613 mg, 87%) as colorless crystals.

A number of embodiments of the invention have been described. Nevertheless, it will be understood that various modifications may be made without departing from the spirit and scope of the invention. Accordingly, other embodiments are within the scope of the following claims.

What is claimed is:

1. A method of making a compound of formula (1): 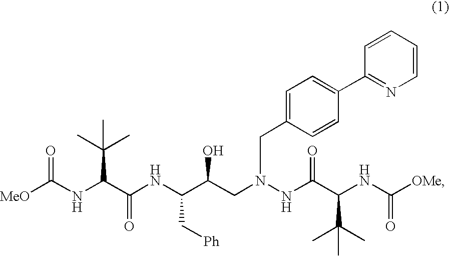
or a salt thereof, the method comprising:
a) reacting a compound of formula (12) or (13): 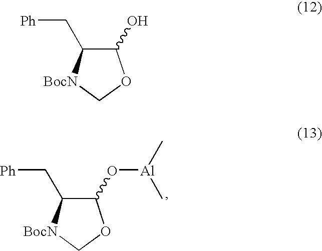
or a salt thereof, with Ar₃P═CH₂, wherein Ar is a substituted or unsubstituted C₄₋₁₂ aryl, followed by acid or base work-up, to produce a compound of formula (7): 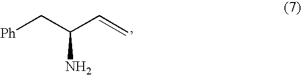
or a salt thereof;
b) coupling the compound of formula (7) with a compound of formula (8): 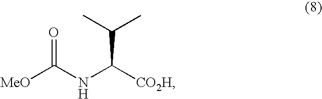
or a salt thereof, to produce a compound of formula (6): 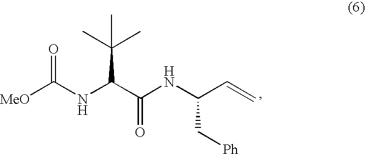
or a salt thereof;
c) reacting the compound of formula (6) with a peroxy acid, or a salt thereof, to produce a compound of formula (2): 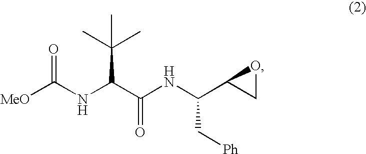
or a salt thereof;
d) reacting 4-(2-pyridyl)benzaldehyde with hydrazine hydrate in a lower alcohol to produce a compound of formula (19): 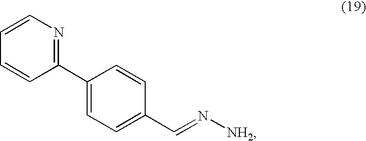
or a salt thereof;
e) coupling the compound of formula (19) with a compound of formula (8): 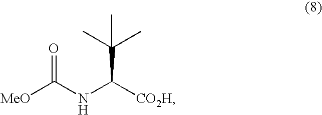
or a salt thereof, to produce a compound of formula (18): 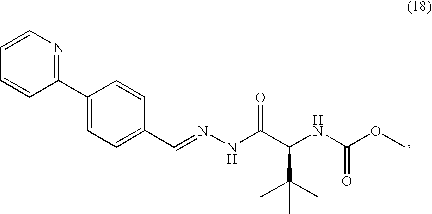
or a salt thereof;
f) hydrogenating the compound of formula (18) with a metal catalyst to produce a compound of formula (3): 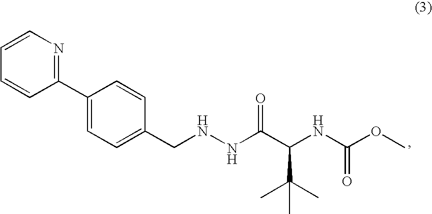
or a salt thereof; and
g) reacting the compound of formula (2), or a salt thereof, with the compound of formula (3), or a salt thereof, in the presence of a Lewis acid catalyst to make the compound of formula (1), or a salt thereof.

2. The method of claim 1, wherein the compound of formula (12), or a salt thereof, is prepared by reacting a compound of formula (15): 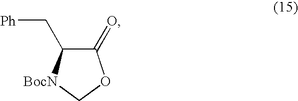
or a salt thereof, with a borohydride.

3. The method of claim 1, wherein the compound of formula (13), or a salt thereof, is prepared by reacting a compound of formula (15): 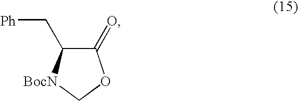
or a salt thereof, with an aluminohydride.

4. The method of claim 2, wherein the borohydride is selected from MBH₄ or MHBR₃; wherein M is selected from Li, Na, K, Bu₄N, Zn or Ca; and R is a C₁₋₁₀ alkyl.

5. The method of claim 4, wherein the borohydride is selected from LiBH₄, NaBH₄, or KBH₄.

6. The method of claim 2, wherein the aluminohydride is selected from HAlR₂, MHAl(OR)₃, and MH₂Al(OR)₂; wherein M is selected from Li, Na, K, Bu₄N, Zn or Ca; and R is a C₁₋₁₀ alkyl.

7. The method of claim 6, wherein the aluminohydride is selected from HAl(Bu-i)₂, LiHAl(O-Bu-t)₃, LiHAl(OMe)₃, LiHAl(OEt)₃, and NaH₂Al(OCH₂CH₂OCH₃)₂.

8. The method of claim 1, wherein Ar is selected from phenyl, anthracyl, and naphthyl.

9. The method of claim 4, wherein Ar is phenyl.

10. The method of claim 1, wherein the coupling is step (b) is performed under standard peptide formation conditions.

11. The method of claim 1, wherein the peroxy acid, or salt thereof, is selected from the group consisting of perbenzoic acid, performic acid, peracetic acid, monoperoxyphthalic acid, pertungstic acid, m-chloroperbenzoic acid, or magnesium bis-monoperoxyphthalate hexahydrate (MMPP).

12. The method of claim 1, wherein the coupling is step (e) is performed under standard peptide formation conditions.

13. The method of claim 1, wherein the metal catalyst in step (f) is selected from: Pd and Ni.

14. The method of claim 13, wherein the metal catalyst is Pd—C.

15. The method of claim 1, wherein the Lewis acid catalyst is a heterogeneous catalyst is selected from: alumina, phosphomolybdic acid-Al₂O₃, Zn(ClO₄)₂—Al₂O₃, SiO₂, heteropolyacids, zirconium sulfophenyl phosphonates, zeolites, clays, mesoporous aluminosilicates, K₅CoW₁₂O₄₀.3H₂O, (NH₄)₈[CeW₁₀O₃₆].20H₂O, Montmorillonite K-10, SBA-15, and Amberlist-15.

16. The method of claim 15, wherein the heterogeneous catalyst is SiO₂.

17. A method of making a compound of formula (1): 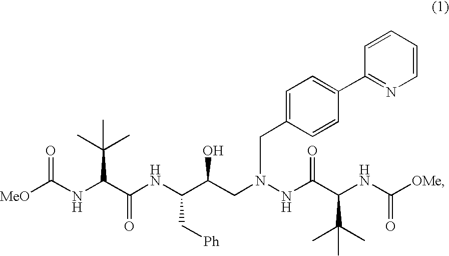
or a salt thereof, the method comprising:
a) reacting a compound of formula (13): 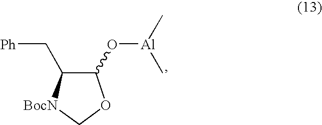
or a salt thereof, with Ph₃P═CH₂ with hydrochloric acid, to produce a compound of formula (7): 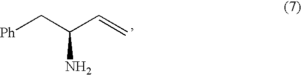
or a salt thereof;
b) coupling the compound of formula (7) with a compound of formula (8): 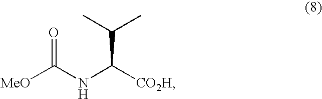
or a salt thereof, to produce a compound of formula (6): 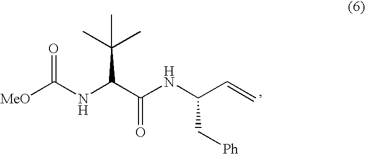
or a salt thereof;
c) reacting the compound of formula (6) with magnesium bis-monoperoxyphthalate hexahydrate (MMPP), to produce a compound of formula (2): 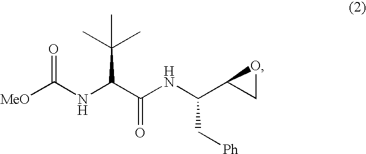
or a salt thereof;
d) reacting 4-(2-pyridyl)benzaldehyde with hydrazine hydrate in a lower alcohol to produce a compound of formula (19): 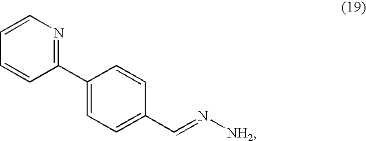
or a salt thereof;
e) coupling the compound of formula (19) with a compound of formula (8): 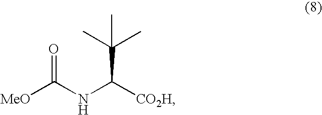
or a salt thereof, to produce a compound of formula (18): 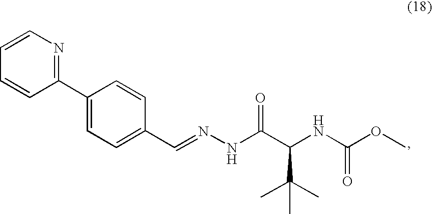
or a salt thereof;
f) hydrogenating the compound of formula (18) with a formate and Pd—C to produce a compound of formula (3): 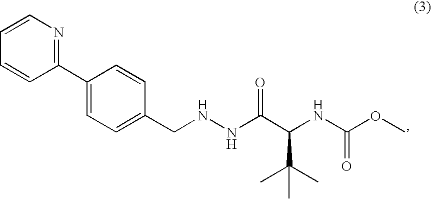
or a salt thereof; and
g) reacting the compound of formula (2), or a salt thereof, with the compound of formula (3), or a salt thereof, in the presence of SiO₂ to make the compound of formula (1), or a salt thereof.

18. The method of claim 17, wherein the formate is selected from sodium formate, potassium formate, and ammonium formate.

19. A method of making a compound of formula (1): 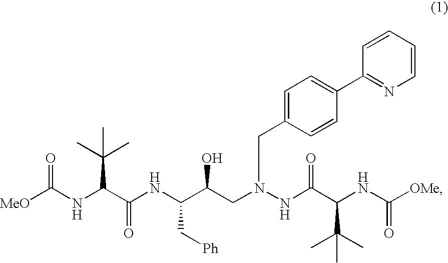
or a salt thereof, the method comprising reacting a compound of formula (2): 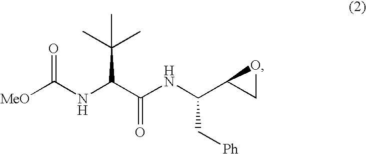
or a salt thereof, with a compound of formula (3): 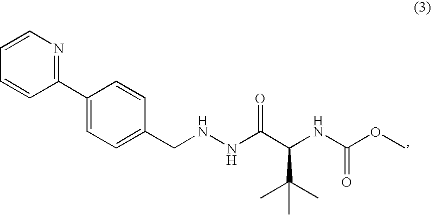
or a salt thereof, in the presence of a Lewis acid catalyst and an inert solvent thereby making the compound of formula (1), or a salt thereof.

20. The method of claim 19, wherein the Lewis acid catalyst is selected from the group consisting of: a metal triflate, metal halide, metal perchlorate, metal tetrafluoroborate, fluoroalkyl alcohol, and a heterogeneous catalyst.

21. The method of claim 20, wherein the metal triflate is selected from the group consisting of: Li(OTf), Sn(OTf)₂, Cu(OTf)₂, Bi(OTf)₃, Ca(OTf)₂, Al(OTf)₃, Sm(OTf)₃, Yb(OTf)₃, and Sc(OTf)₃.

22. The method of claim 20, wherein the metal halide is selected from the group consisting of: CeCl₃, WCl₆, ZrCl₄, RuCl₃, SbCl₃, CoCl₂, CdCl₂, TaCl₅, InCl₃, BiCl₃, VCl₃, SnCl₄, ZnCl₂, ZrCl₄, InBr₃, MgBr₂, LiBr, SmI₂, and SmCl₃.

23. The method of claim 20, wherein the metal perchlorate is selected from the group consisting of: LiClO₄, NaClO₄, Zn(ClO₄)₂, and Cu(ClO₄)₂.

24. The method of claim 20, wherein the fluoroalkyl alcohol is hexafluoro-2-propanol.

25. The method of claim 20, wherein the Lewis acid is a heterogeneous catalyst selected from the group consisting of: alumina, phosphomolybdic acid-Al₂O₃, Zn(ClO₄)₂—Al₂O₃, SiO₂, heteropolyacids, zirconium sulfophenyl phosphonates, zeolites, clays, mesoporous aluminosilicates, K₅CoW₁₂O₄₀.3H₂O, (NH₄)₈[CeW₁₀O₃₆].20H₂O, Montmorillonite K-10, SBA-15, and Amberlist-15.

26. The method of claim 19, wherein the Lewis acid catalyst is SiO₂.

27. The method of claim 19, wherein the inert solvent is selected from the group consisting of: an alcohol, ether, amide, ester, chlorinated hydrocarbon, hydrocarbon, or mixtures thereof.

28. The method of claim 27, wherein the alcohol is selected from the group consisting of: methanol, ethanol, n-propanol, isopropanol, n-butanol, isobutanol, tert-butanol, and ethylene glycol.

29. The method of claim 27, wherein the ether is selected from the group consisting of: diethyl ether, diisopropyl ether, tetrahydrofuran, and dioxane.

30. The method of claim 27, wherein the amide is selected from the group consisting of: dimethylformamide and dimethylacetamide.

31. The method of claim 27, wherein the ester is selected from the group consisting of: ethyl acetate, methyl acetate and ethyl formate.

32. The method of claim 27, wherein the chlorinated hydrocarbon is selected from the group consisting of: dichloromethane, chloroform, and dichloroethane.

33. The method of claim 27, wherein the hydrocarbon is selected from the group consisting of: hexane, heptane, benzene, and toluene.

34. The method of claim 19, wherein the inert solvent is dichloromethane.

35. A method of making a compound of formula (4): 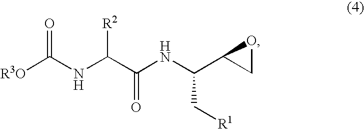
or a salt thereof, wherein:
R¹ is selected from C₄₋₁₀ alkyl, C₅₋₁₂ cycloalkyl, C₅₋₁₂ heterocycloalkyl, C₅₋₁₂ aryl, C₅₋₁₂ heteroaryl, and an amino acid side chain, wherein the alkyl, cycloalkyl, heterocycloalkyl, aryl and heteroaryl can be substituted or unsubstituted; and
R² is selected from H, C₁₋₁₀ substituted or unsubstituted alkyl, and an amino acid side chain; and
R³ is selected from H and C₁₋₁₀ substituted or unsubstituted alkyl;
the method comprising: reacting a compound of formula (5): 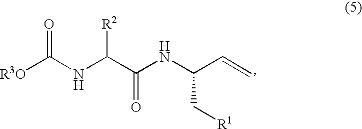
or a salt thereof, wherein R¹, R², and R³ are as defined above;
with one or more of:
a) molecular oxygen or air in the presence of a metal catalyst;
b) hydrogen peroxide or an organic peroxide in the presence of a metal catalyst; or
c) a peroxy acid, or a salt thereof;
in a solvent, thereby making a compound of formula (4), or a salt thereof.

36. The method of claim 35, wherein the metal catalyst is selected from: a transition metal oxide solid acid (TMO); tungstic acid or its derivatives; vanadium complexes; molybdenum complexes; titanium complexes; and cobalt complexes.

37. The method of claim 35, wherein the peroxy acid, or salt thereof, is selected from the group consisting of perbenzoic acid, performic acid, peracetic acid, monoperoxyphthalic acid, pertungstic acid, m-chloroperbenzoic acid, or magnesium bis-monoperoxyphthalate hexahydrate (MMPP).

38. The method of claim 36, wherein the peroxy acid is MMPP.

39. The method of claim 35, wherein the solvent is selected from the group consisting of: alcohols, ethers, or chlorinated hydrocarbons.

40. The method of claim 39, wherein the solvent is dichloromethane.

41. The method of claim 39, wherein the solvent is methanol.

42. The method of claim 35, wherein R¹ is a C₅₋₁₂ aryl.

43. The method of claim 35, wherein R¹ is an amino acid side chain selected from a side chain of histidine, phenylalanine, tryptophan, and tyrosine.

44. The method of claim 42, wherein R¹ is phenyl.

45. The method of claim 44, wherein R² is an amino acid side chain.

46. The method of claim 35, wherein R² is a C₁₋₁₀ alkyl.

47. The method of claim 46, wherein R² is tert-butyl.

48. The method of claim 35, wherein R³ is a C₁₋₁₀ alkyl.

49. The method of claim 48, wherein R³ is methyl.

50. The method of claim 35, wherein the compound of formula (4) is: 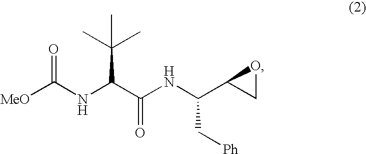
or a salt thereof.

51. A method of making a compound of formula (2): 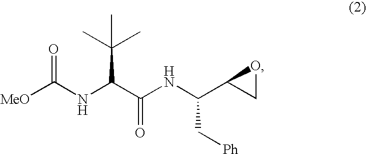
or a salt thereof, the method comprising:
reacting a compound of formula (6): 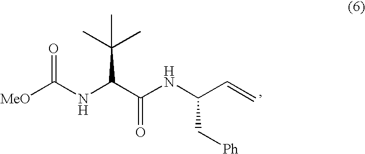
or a salt thereof, with one or more of:
a) molecular oxygen or air in the presence of a metal catalyst;
b) hydrogen peroxide or an organic peroxide in the presence of a metal catalyst; or
c) a peroxy acid, or a salt thereof;
in a solvent, thereby making a compound of formula (2), or a salt thereof.

52. A method of making a compound of formula (16): 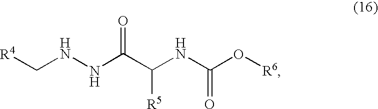
or a salt thereof, wherein:
R⁴ is selected from C₄₋₁₀ alkyl, C₅₋₁₂ cycloalkyl, C₅₋₁₂ heterocycloalkyl, C₅₋₁₂ aryl, C₅₋₁₂ heteroaryl, and an amino acid side chain, wherein the alkyl, cycloalkyl, heterocycloalkyl, aryl and heteroaryl can be substituted or unsubstituted; and
R⁵ is selected from H, C₁₋₁₀ substituted or unsubstituted alkyl, and an amino acid side chain; and
R⁶ is selected from H and C₁₋₁₀ substituted or unsubstituted alkyl;
the method comprising reacting a compound of formula (17): 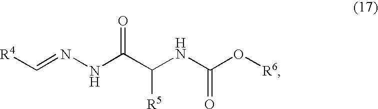
or a salt thereof, wherein R⁴, R⁵, and R⁶ are as defined above;
by one of the following:
a) reducing the compound of formula (17) with a hydride in the presence of an acid;
b) hydrogenating the compound of formula (17) with a metal catalyst; or
c) transfer hydrogenating the compound of formula (17) with a metal catalyst;
thereby making a compound of formula (16), or a salt thereof.

53. The method of claim 52, wherein the hydride is a borohydride.

54. The method of claim 53, wherein the borohydride is selected from LiBH₄, NaBH₄, KBH₄, of NaBH₃CN.

55. The method of claim 54, wherein the borohydride is NaBH₃CN.

56. The method of claim 52, wherein the acid is a sulphonic acid.

57. The method of claim 55, wherein the sulphonic acid is selected from: p-toluenesulphonic acid, methanesulphonic acid, and benzenesulphonic acid.

58. The method of claim 57, wherein the sulphonic acid is p-toluenesulphonic acid.

59. The method of claim 52, wherein the metal catalyst is Pd or Ni.

60. The method of claim 59, wherein the metal catalyst is Pd—C.

61. The method of claim 52, wherein the transfer hydrogenation further comprises a formate as a hydrogen source.

62. The method of claim 52, wherein R⁴ is a C₅₋₁₂ aryl.

63. The method of claim 62, wherein R⁴ is 4-(2-pyridyl)phenyl.

64. The method of claim 52, wherein R⁵ is an amino acid side chain.

65. The method of claim 52, wherein R⁵ is a C₁₋₁₀ alkyl.

66. The method of claim 65, wherein R⁵ is tert-butyl.

67. The method of claim 52, wherein R⁶ is a C₁₋₁₀ alkyl.

68. The method of claim 67, wherein R⁶ is methyl.

69. The method of claim 52, wherein the compound of formula (16) is: 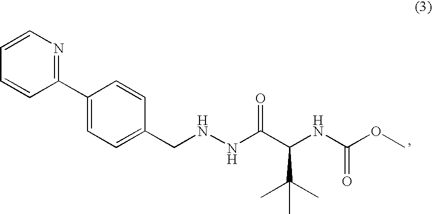
or a salt thereof.

70. A method of making a compound of formula (3): 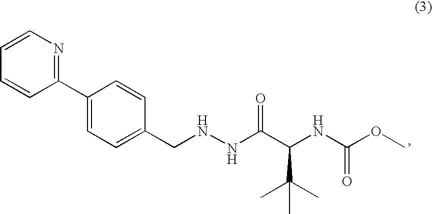
or a salt thereof; the method comprising reacting a compound of formula (18): 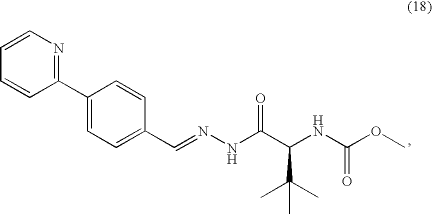
or a salt thereof, by one of the following:
a) reducing the compound of formula (18) with a hydride in the presence of an acid;
b) hydrogenating the compound of formula (18) with a metal catalyst; or
c) transfer hydrogenating the compound of formula (18) with a metal catalyst;
thereby making a compound of formula (3), or a salt thereof.

71. A method of making a compound of formula (9): 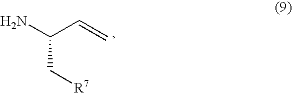
or a salt thereof, wherein:
R⁷ is a C₄₋₁₀ alkyl, C₅₋₁₂ cycloalkyl, C₅₋₁₂ heterocycloalkyl, C₅₋₁₂ aryl, C₅₋₁₂ heteroaryl, and an amino acid side chain, wherein the alkyl, cycloalkyl, heterocycloalkyl, aryl and heteroaryl can be substituted or unsubstituted;
the method comprising reacting a compound of formula (10) or (11): 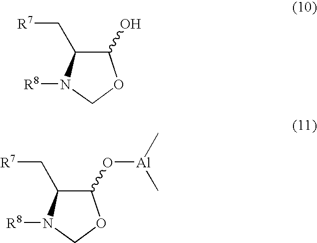
or a salt thereof, wherein:
R⁷ is as defined above; and
R⁸ is an amino protecting group;
with one of the following:
a) Ar₃P═CH₂, wherein Ar is an unsubstituted or substituted aryl or heteroaryl;
b) CH₂I₂—Zn—AlMe₃, followed by acid or base work-up; or
c) a compound of formula (14): 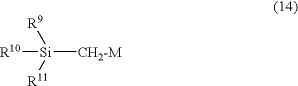
wherein:
R⁹, R¹⁰, and R¹¹ are independently a substituted or unsubstituted C₁₋₁₀ alkyl or C₅₋₁₂ aryl; and
M is Li, MgCl, MgBr, or MgI;
followed by treatment with a strong acid;
thereby making a compound of formula (9) or a salt thereof.

72. The method of claim 71, wherein R⁷ is an amino acid side chain.

73. The method of claim 71, wherein the amino acid side chain is selected from a side chain of histidine, phenylalanine, tryptophan, and tyrosine.

74. The method of claim 71, wherein R⁷ is a C₅₋₁₂ aryl.

75. The method of claim 73, wherein R⁷ is phenyl.

76. The method of claim 71, wherein the amino protecting group is selected from alkyloxycarbonyl, triarylmethyl, tert-butyloxycarbonyl (Boc), or triphenylmethyl (Tr).

77. The method of claim 76, wherein the amino protecting group is tert-butyloxycarbonyl.

78. The method of claim 71, wherein Ar is selected from phenyl, anthracyl, and naphthyl.

79. The method of claim 75, wherein Ar is phenyl.

80. The method of claim 71, wherein reaction a) is conducted under standard Wittig reaction conditions.

81. The method of claim 71, wherein the strong acid is selected from BF₃ Et₂O, H₂SO₄, or HO₂CCF₃.

82. The method of claim 71, wherein the compound of formula (9) is: 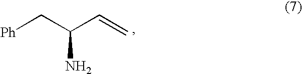
or a salt thereof.

83. A method of making a compound of formula (7): 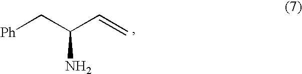
or a salt thereof, the method comprising reacting a compound of formula (12) or (13): 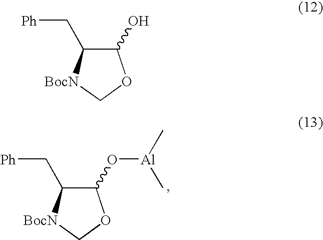
or a salt thereof, with one of the following:
a) Ar₃P═CH₂, wherein Ar is an unsubstituted or substituted aryl or heteroaryl;
b) CH₂I₂—Zn—AlMe₃, followed by acid or base work-up; or
c) a compound of formula (14): 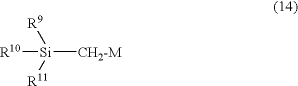
wherein:
R⁹, R¹⁰, and R¹¹ are independently a substituted or unsubstituted C₁₋₁₀ alkyl or C₅₋₁₂ aryl; and
M is Li, MgCl, MgBr, or MgI;
followed by treatment with a strong acid;
thereby making a compound of formula (7) or a salt thereof.

84. A compound according to formula (16) 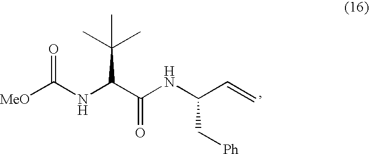
or a salt thereof.

85. A compound according to formula (2): 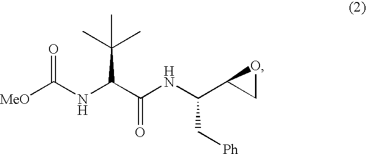
or a salt thereof.

86. A composition comprising a carrier and a compound according to formula (16) 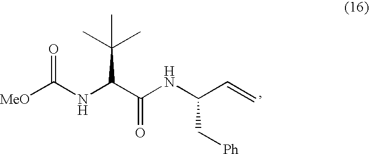
or a salt thereof.

87. A composition comprising a carrier and a compound according to formula (2): 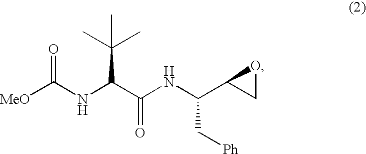
or a salt thereof.

88. The composition of claim 86, wherein the carrier is a pharmaceutically acceptable carrier.

89. The composition of claim 87, wherein the carrier is a pharmaceutically acceptable carrier.
